(19) United States
(12) Patent Application Publication
(10) Pub. No.: US 2026/0295225 A1
(43) Pub. Date: Oct. 1, 2026

(54) Lumen Cross-Sectional Fitting Dilator

(71) Applicant: KDS Innovations, LLC, Clear Lake, MN (US)

(72) Inventors: Thom Dahle, Clear Lake, MN (US); Stephen Kidd, St. Cloud, MN (US); Brian Stegman, Sartell, MN (US)

(73) Assignee: KDS Innovations, LLC, Clear Lake, MN (US)

(21) Appl. No.: 19/629,593

(22) Filed: Mar. 26, 2026

Related U.S. Application Data
(60) Provisional application No. 63/778,134, filed on Mar. 26, 2025.

(51) Int. Cl. A61M 29/00

(52) U.S. Cl. CPC A61M 29/00; A61M 2210/12

(57) ABSTRACT

Apparatus and related methods generally relate to a lumen-fitted dilator (LFD). In an illustrative example, the LFD may have an exterior surface configured to slidingly interface with a catheter (e.g., a guide extension catheter (GEC)) such that a fitted transition is formed between the exterior surface of the LFD and a distal mouth of a lumen of the catheter. The LFD may, for example, have adaptive geometries configured to interface with multiple GECs. The LFD may, for example, have dedicated geometries configured to interface with a special-purpose GEC, such as a dilator-fitted GEC (DFGEC). Various embodiments may advantageously reduce or eliminate fishmouthing between a dilator and GEC (e.g., 5-7 French), which may, for example, advantageously enhance passage and/or reduce ‘snagging’ of a catheter such as during vascular procedures (e.g., peripheral vascular procedures and/or coronary artery intervention).

### CROSS-REFERENCE TO RELATED APPLICATIONS

This application claims the benefit of U.S. Provisional Application 63/778,134, filed Mar. 26, 2025 by Dahle, Kidd, and Stegman, titled “Lumen Cross-Sectional Fitting Dilator.” This application has common inventors and/or related subject matter with U.S. Provisional Application 63/655,044, filed Jun. 2, 2024 by Dahle, Stegman, and Kidd, titled “Devices, Systems, and Methods for Crossing Complex Vascular Anatomy.” The entire contents of each of the foregoing applications and their priority applications, if any, are incorporated herein by reference.

Unless expressly stated, changes in terminology from priority application(s) to this application are made without prejudice or disclaimer of subject matter. Changes from the priority application(s) (e.g., provisional applications(s)) are intended to be broadening and/or additive unless expressly stated otherwise. Replacement of alternative terms with a single representative term, for example, are inclusive unless otherwise defined. Various embodiments may also be found in previous disclosure(s) incorporated by reference. Embodiments of similar languages in this application are not modifications or disclaimer of the embodiments disclosed in previous incorporated disclosures unless otherwise stated.

### BACKGROUND

Catheters can be inserted through different body pathways, such as to access specific anatomical regions, for example. This flexibility makes them widely applicable for multiple different medical procedures. Such medical procedures may include, for example, fluid drainage, medication delivery, and/or diagnostic imaging across various clinical settings. For example, catheters may be used in urological, cardiovascular, and/or interventional procedures. Catheter types may include, for example, Foley catheters (e.g., which may be used for urinary drainage), central venous catheters (e.g., which may be used for long-term intravenous access), and peripheral intravenous catheters (e.g., which may be used for short-term medication administration). Healthcare professionals such as physicians may, for example, utilize catheters in numerous medical specialties, including, by way of example and not limitation, oncology, nephrology, and/or emergency care, such as to manage patient conditions, monitor physiological parameters, and/or deliver targeted treatments with minimal invasiveness.

Guide extension catheters (GECs) represent specialized medical instruments. GECs may, for example, often be employed in interventional radiology and/or endovascular procedures. These catheters may, for example, be designed to provide enhanced navigational support and/or access to challenging anatomical locations during complex medical interventions. GECs may, for example, be used in neurovascular, peripheral vascular, and/or coronary interventions. In such applications, guide extension catheters may be configured, for example, to enable physicians to reach difficult-to-access regions with improved precision and/or control. They may, for example, be particularly valuable in procedures such as angioplasty, stent placement, and/or embolization, where precise catheter positioning may be key to successful treatment outcomes.

Dilators include medical devices configured to gradually expand and/or widen narrow anatomical passages. Dilators may, for example, advantageously facilitate access for diagnostic and/or therapeutic procedures. Dilators may, for example, be employed across multiple medical disciplines, including urology, interventional radiology, and/or vascular surgery. In vascular access procedures, dilators may, for example, be configured to create controlled pathways for catheter insertion, such as by progressively enlarging vessel entry points. Physicians may, for example, advantageously utilize dilators in techniques such as percutaneous transluminal angioplasty, central venous catheterization, and/or urological interventions, where controlled tissue expansion may help minimize trauma and/or enable safe, effective medical access.

### TECHNICAL FIELD

Apparatus and methods generally relate to catheters.

### BRIEF SUMMARY

Apparatus and related methods generally relate to a lumen-fitted dilator (LFD). In an illustrative example, the LFD may have an exterior surface configured to slidingly interface with a catheter (e.g., a guide extension catheter (GEC)) such that a fitted transition is formed between the exterior surface of the LFD and a distal mouth of a lumen of the catheter. The LFD may, for example, have adaptive geometries configured to interface with multiple GECs. The LFD may, for example, have dedicated geometries configured to interface with a special-purpose GEC, such as a dilator-fitted GEC (DFGEC). Various embodiments may advantageously reduce or eliminate fishmouthing between a dilator and GEC (e.g., 5-7 French), which may, for example, advantageously enhance passage and/or reduce ‘snagging’ of a catheter such as during vascular procedures (e.g., peripheral vascular procedures and/or coronary artery intervention).

Further variations and embodiments will be apparent from the detailed description.

### BRIEF DESCRIPTION OF THE DRAWINGS

Various embodiments of the present embodiments are described with reference to the following FIGURES.

FIG. 1 depicts an example guide extension catheter (GEC) lumen-fitted dilator (LFD) in an illustrative use-case scenario in a tortuous vasculature.

FIG. 2 depicts an example LFD contrasted to a traditional dilator with a gaping mouth formed by a loose fit between the GEC lumen and the outer diameter (OD) of the dilator.

FIG. 3 depicts the example LFD of FIG. maintaining a smooth transition from dilator to GEC in an illustrative configuration such as the GEC-LFD assembly may be operated into while traversing tortuous passages.

FIG. 4 depicts an illustrative LFD assembled within an illustrative dilator-fitting GEC (DFGEC).

FIG. 5 depicts an illustrative LFD with adjustable lumen fitment.

FIG. 6A and FIG. 6B depict cross-section views along a longitudinal axis of an illustrative self-adjusting outer diameter LFD (SAODLFD).

FIG. 6C is a cross-section view of a SAODLFD across the longitudinal axis.

FIG. 6D is a cross-section view showing support elements configured as longitudinally-extending ribs.

FIG. 6E is a cross-section view showing illustrative dimensions.

FIG. 7A depicts an illustrative LFD with a selectively-actuated adjustable lumen fitment module.

FIG. 7B depicts an example LFD with a lumen fitment module configured as a hub sized and positioned to limit axial advancement of the LFD through a GEC.

FIG. 7C depicts a cross-section view along a longitudinal axis of the assembled LFD with lumen fitment module of FIG. B in an illustrative use case in which the LFD is inserted into a GEC.

FIG. 8 depicts an illustrative longitudinally-relieved LFD.

FIG. 9 depicts an illustrative cross-section view along the longitudinal axis of the longitudinally-relieved LFD, in an embodiment including a lumen access port and a circumferential axial translation module.

FIG. 10 depicts a view from the distal end towards the proximal end across the longitudinal axis of the LFD depicted in FIGS. 8-9.

FIG. 11 depicts illustrative cross-sectional geometry across the longitudinal axis of example LFDs.

FIG. 12 depicts an LFD operably coupled to a separate circumferential engagement translation module (CETM).

FIG. 13 depicts a DFGEC with integrated axial interdigitation module operably engaging an axial interdigitation module of an LFD.

FIG. 14 depicts a cross-section view along the longitudinal axis of the view of FIG. 13.

FIG. 15 depicts an illustrative LFD with a stepped-diameter distal tip and an example DFGEC with integrated axial interdigitation module.

FIG. 16 depicts an illustrative CETM which may be integrated with the GEC body.

FIG. 17 depicts an illustrative CETM which may be coupled to the proximal end of the GEC.

FIG. 18 depicts an example CETM coupled to the proximal end of the GEC.

FIG. 19 depicts an example CETM configured to receive an illustrative planar-assembled axial translation member.

FIG. 20 depicts a reduced-clearance DFGEC assembly including the CETM of FIG. 19 assembled substantially in-plane with an axial translation member.

FIG. 21 depicts a dilator with a lumen access port.

FIG. 22 depicts a method of deploying an embedded device in the body.

FIG. 23A, FIG. 23B, and FIG. 24 depict illustrative fenestrated GECs.

FIG. 25 depicts an illustrative LFD provided with a lumen-dilating module in an illustrative assembly.

FIG. 26A and FIG. 26B depict variations of the illustrative LFD.

FIG. 27 depicts an illustrative LFD being inserted into a GEC.

FIG. 28 depicts an illustrative LFD being inserted into a clear GEC (reinforced with an embedded coil, as depicted).

FIG. 29 depicts an illustrative embodiment of an LFD with axial engagement module. The LFD includes an integrally molded axial engagement module.

FIG. 30 depicts an illustrative implementation (shortened for ease of viewing) of a DFGEC.

FIG. 31 depicts illustrative views of the embodiment depicted in FIG. 30.

FIG. 32 depicts an illustrative implementation (cross-section view along the longitudinal axis, and shortened for ease of depiction) of a DFGEC and GEC-matched LFD (e.g., such as disclosed at least with reference to FIG. 5, FIGS. 7B-7C).

FIG. 33 depicts an illustrative prototype of an LFD with a lumen access port, such as disclosed at least with reference to FIGS. 9 and 22.

FIG. 34 depicts an illustrative proportional relationship of a dilator (left two portions) and GEC (right portion).

FIG. 35 depicts illustrative implementations of a cross-section of a dilator comparing different example taper angles.

FIG. 36 depicts a dilator disposed (e.g., overmolded) on an axial translation member.

Like reference numerals refer to like parts throughout the various views unless otherwise specified. Embodiments and portions of embodiments illustrated and described herein are non-limiting and non-exhaustive.

### DETAILED DESCRIPTION

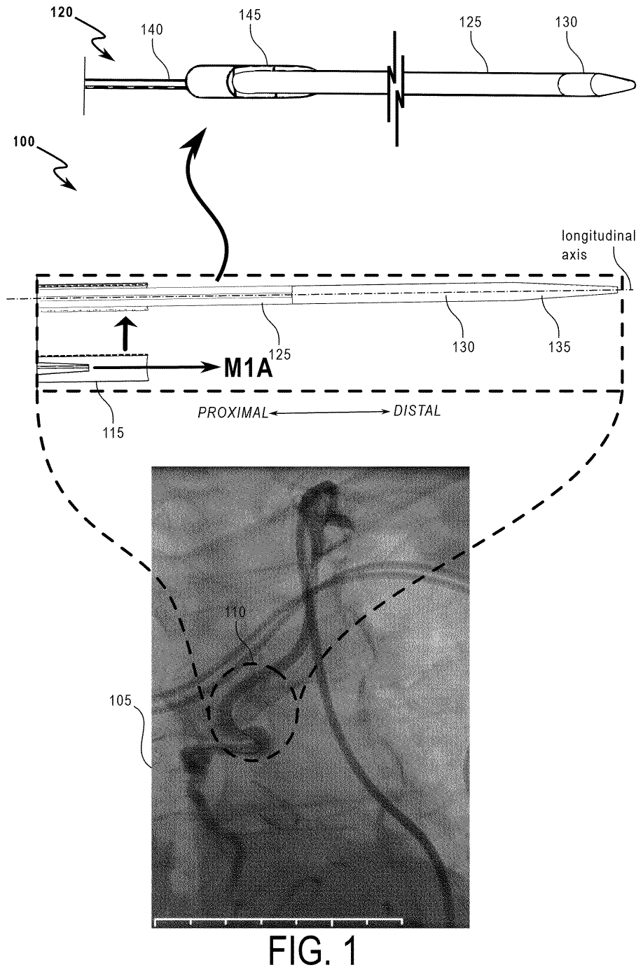

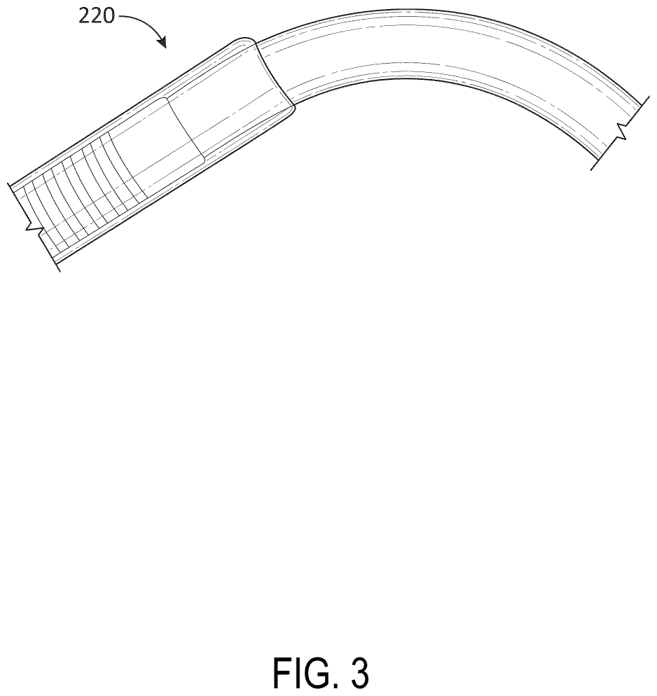

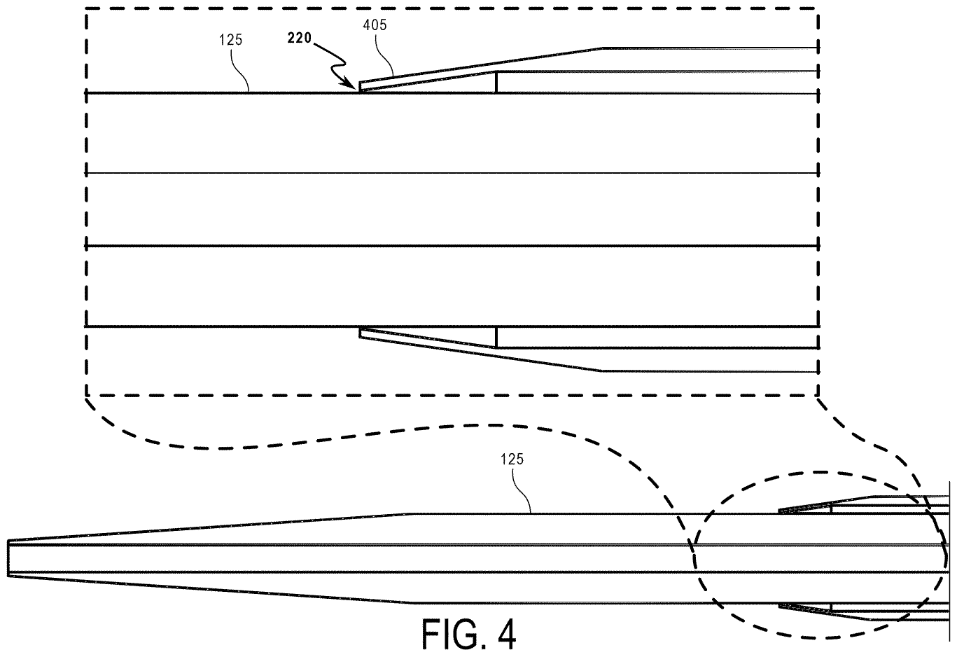

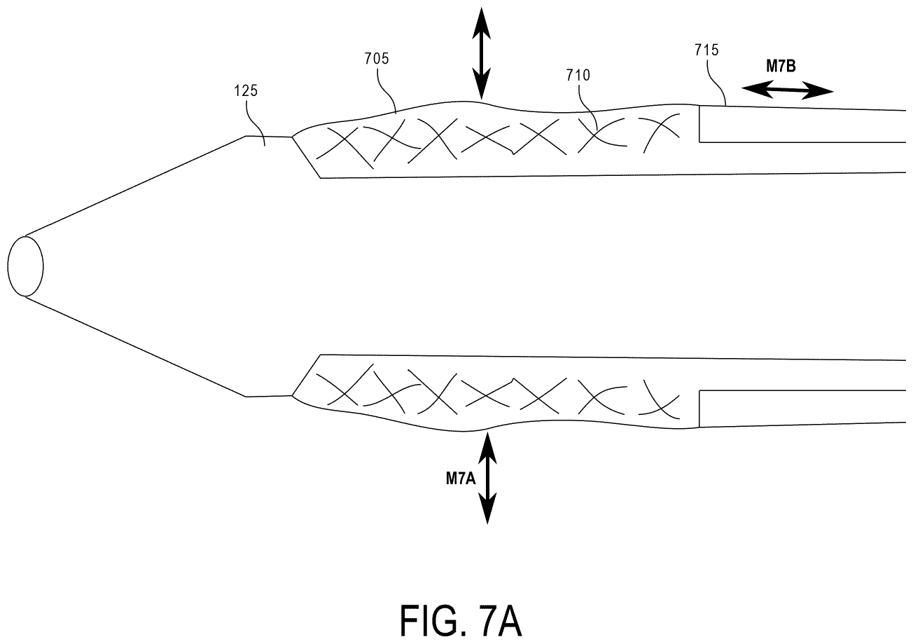

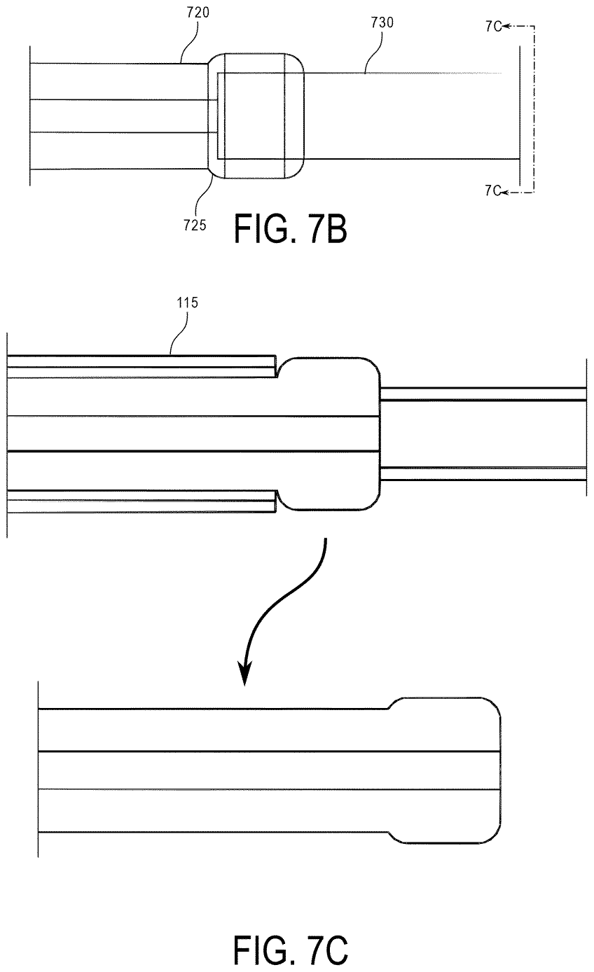

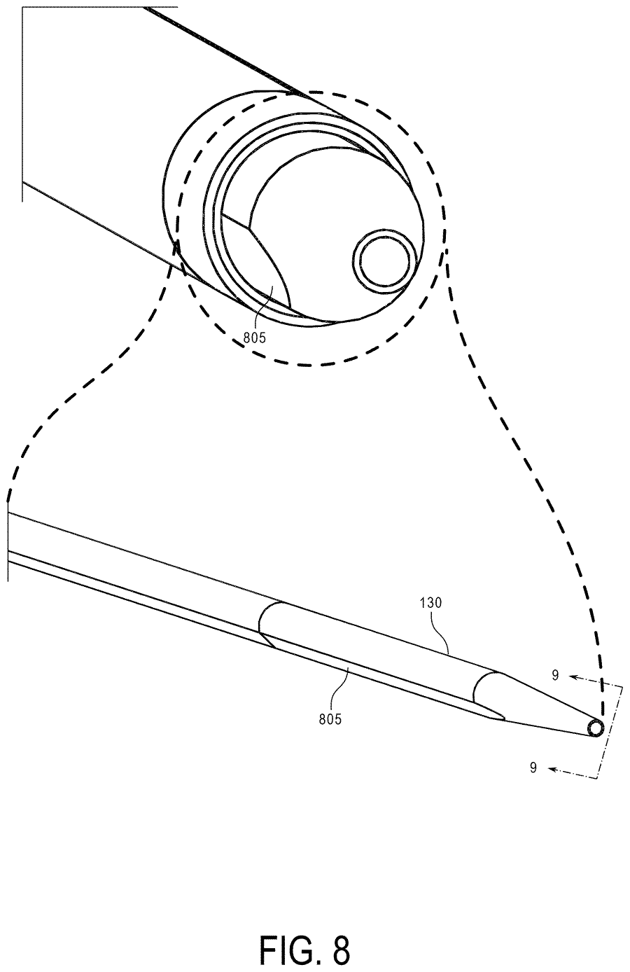

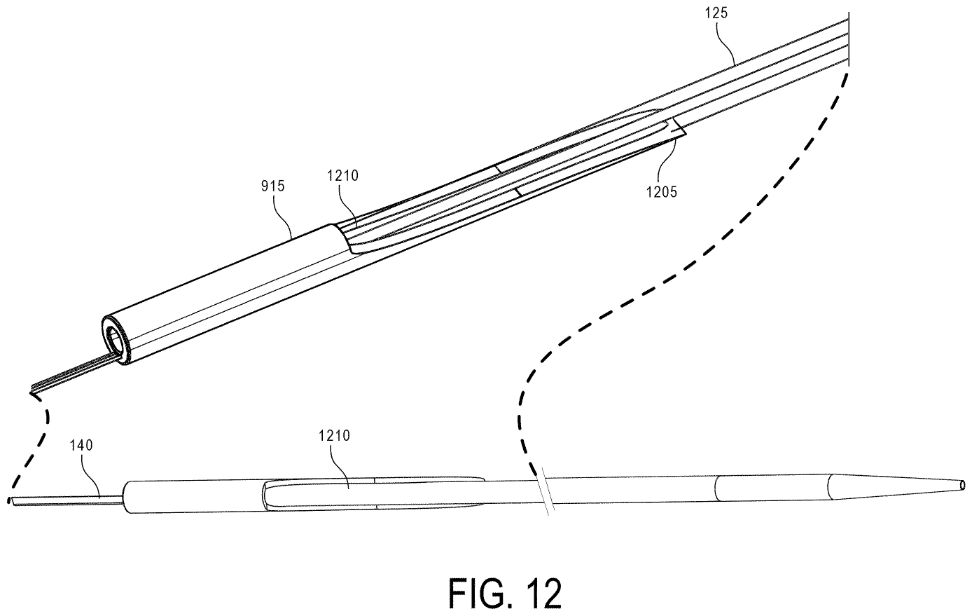

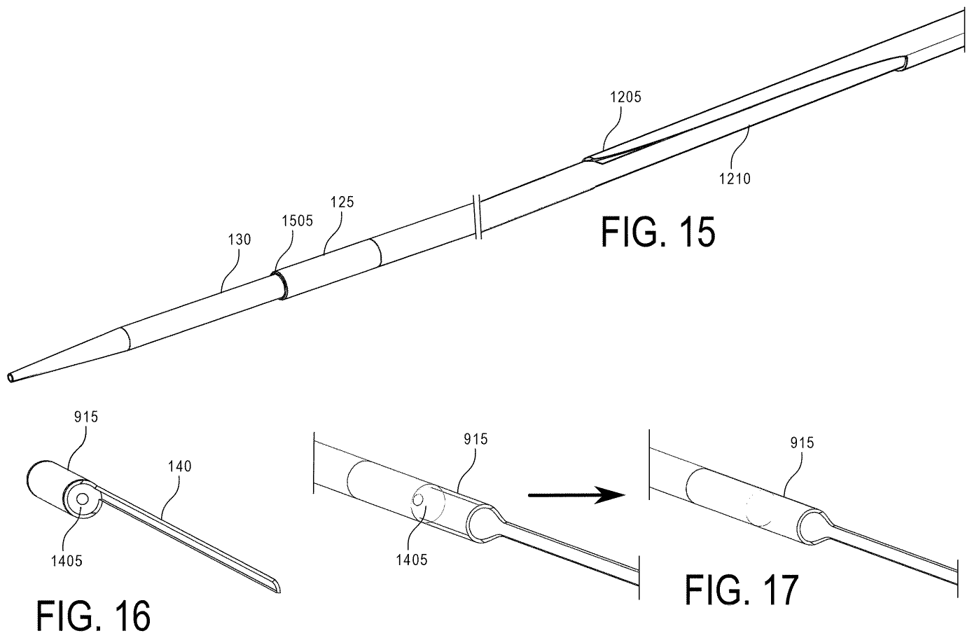

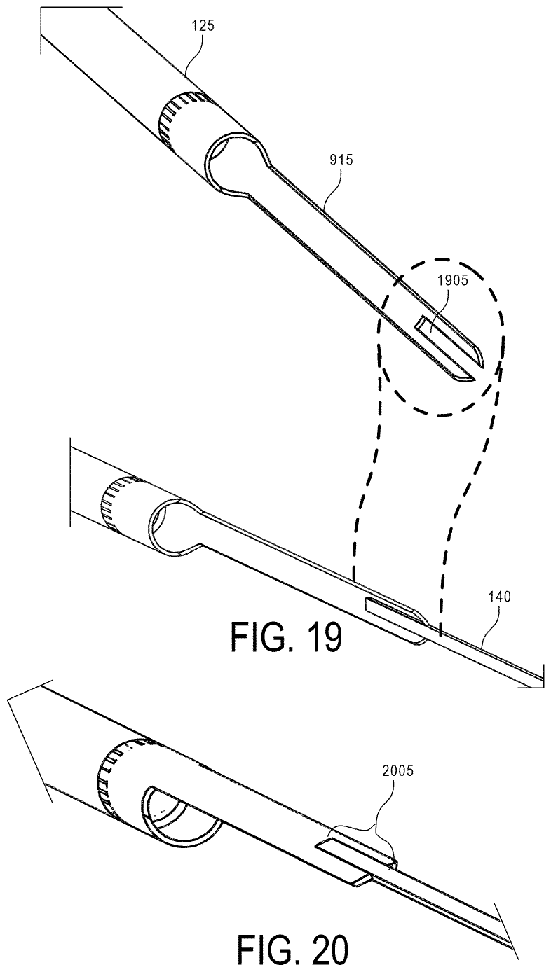

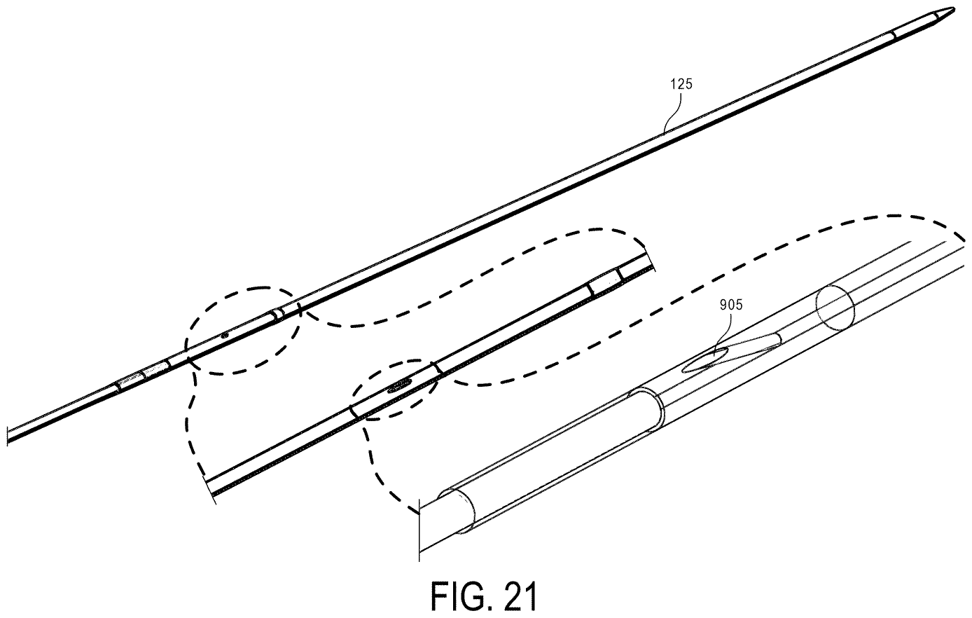

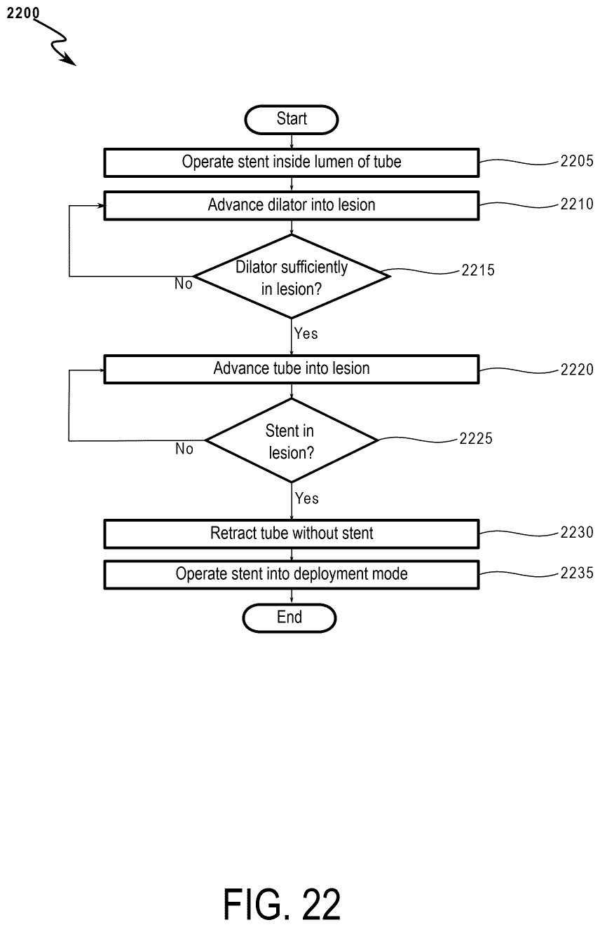

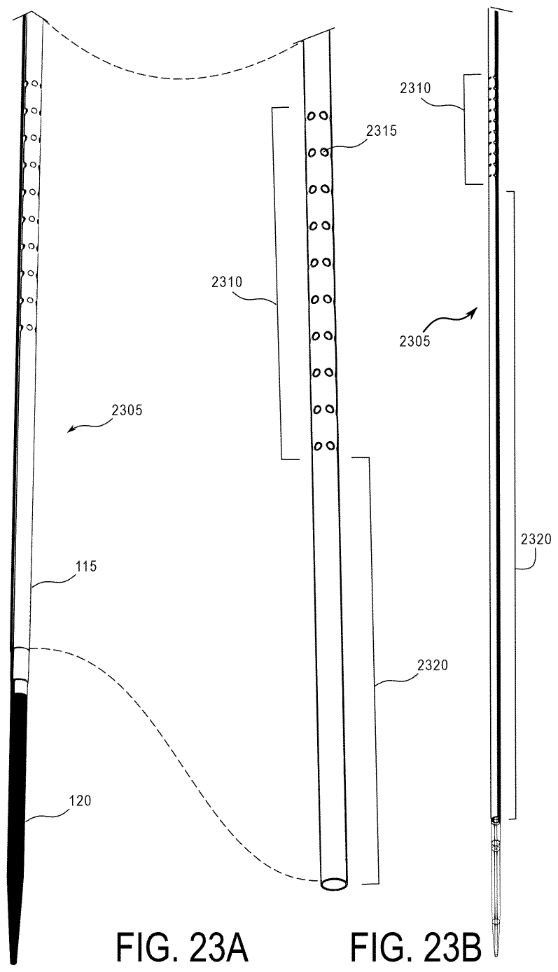

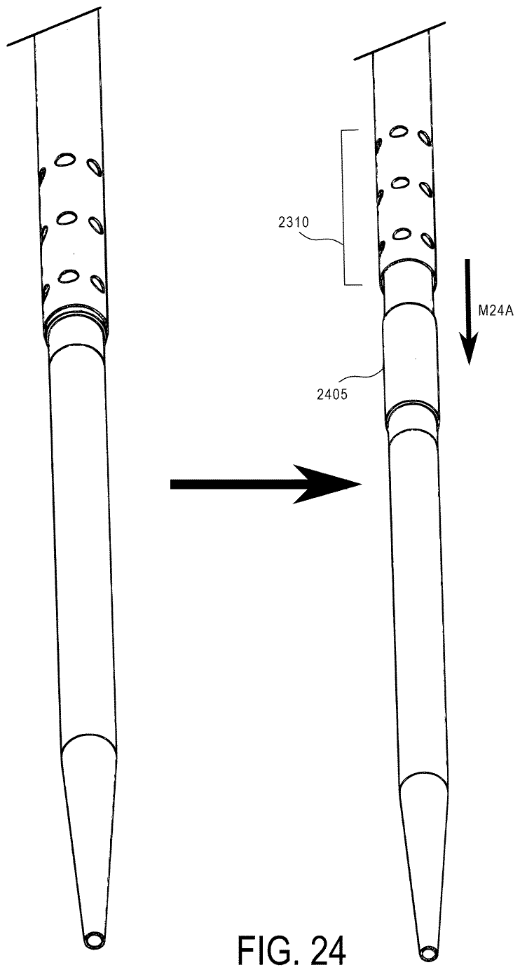

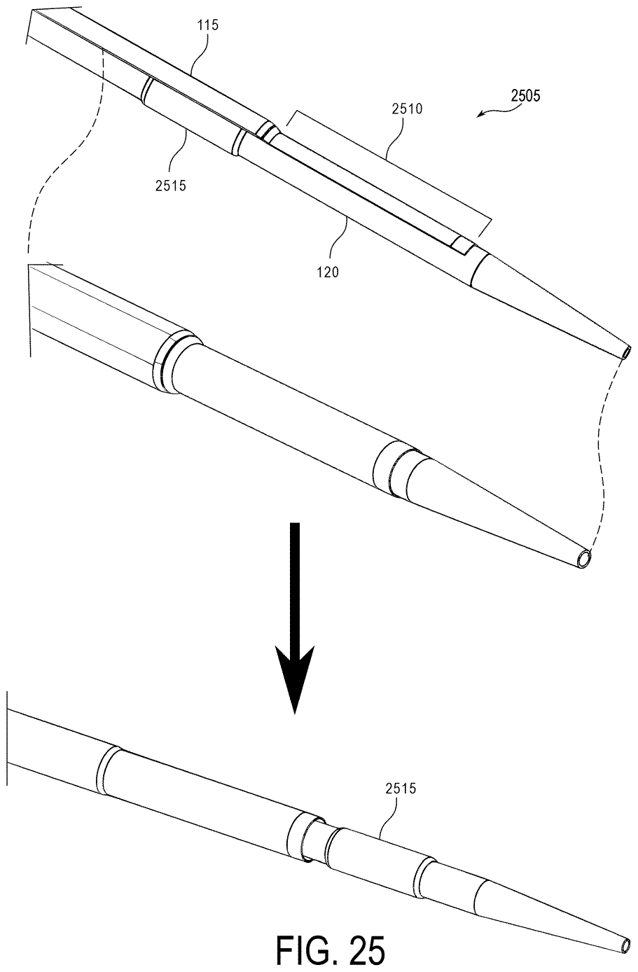

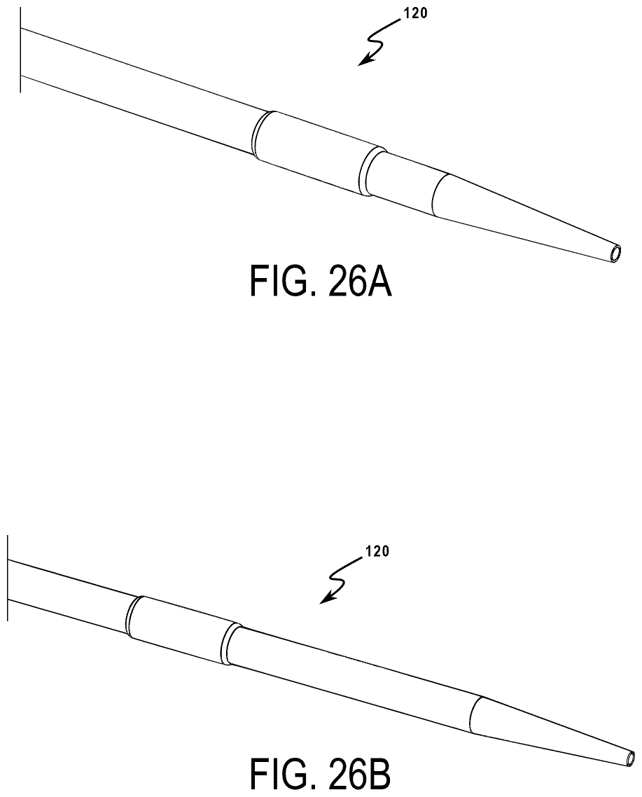

In order to assist rapid comprehension, this document introduces a lumen-fitted dilator (LFD) configured to reduce or prevent fish-mouthing when disposed within a guide extension catheter (GEC) in FIGS. 1-3. Some example configurations of LFDs are disclosed with respect to FIGS. 3-22, especially relating to providing a reduced clearance between the LFD and a corresponding GEC. By way of example and not limitation, some self-adjusting outer diameter configurations are described regarding FIGS. 4-6E. The discussion turns to selectively-actuated adjustable lumen fitment modules with reference to FIG. 7A. Further embodiments configured to provide a smooth transition from a dilator to a proximal end of a GEC are disclosed at least with reference to FIGS. 7B-7C. Then, longitudinally-relieved LFD configurations are disclosed regarding FIGS. 8-11. Examples of LFD circumferential engagement translation modules are introduced with reference to FIGS. 12-14. With respect to FIGS. 15-18, this document discusses some example stepped-diameter and push member (e.g., integrated) configurations. Reduced clearance push rod configurations are discussed in relation to FIGS. 19-20. Lumen access port embodiments are described with reference to FIG. 21. Example methods of use of a LFD to deploy a medical device (e.g., a stent) is disclosed relative to FIG. 22. Fenestrated GEC embodiments and methods are disclosed with reference to FIGS. 23A-24. Features related to lumen-expanding dilators and associated GECs are discussed with reference to FIGS. 25-26. Finally, various additional embodiments and features are discussed related to LFDs and methods of use.

FIG. 1 depicts an example guide extension catheter (GEC) lumen-fitted dilator (LFD) in an illustrative use-case scenario in a tortuous vasculature. In this example 100, a blockage 105 is present in a tortuous region of vasculature 100. A guide extension catheter 115 is deployed in the vasculature 100. A dilator 120 is disposed within the GEC 115. The dilator 120 may, for example, be configured with a snug sliding fit within the GEC 115. For example, the snug fit may advantageously be configured to prevent fish-mouthing at a transition point where the dilator 120 extends from the distal end of the GEC 115, such as when the dilator is advanced (motion MIA) out of the GEC 115.

A smooth, fitted transition from dilator to GEC may, for example, advantageously enhance tracking performance through complex vascular anatomies. A fitted interface between the dilator and GEC may, for example, advantageously reduce risk of vessel wall trauma, such as by eliminating irregular contact points, for example. The maintained coaxial alignment between the dilator and GEC may, for example, advantageously improve precision during interventional procedures. Additional advantages may, for example, include reduced friction during catheter manipulation, improved subsequent device deployment, and/or enhanced overall procedural efficiency and reliability. One or more of these illustrative advantages may, by way of example and not limitation, be achieved through one or more of the example LFD embodiments disclosed herein.

The LFD configurations disclosed herein may, for example, advantageously address multiple challenges associated with fishmouthing in guide extension catheters. For example, the fitted transition between the LFD and GEC may help maintain structural integrity during advancement through tortuous anatomy. The maintained structural integrity may, for example, advantageously provide improved pushability and/or positional stability during interventional procedures. For example, a snug fit without ‘jamming’ the dilator in the GEC may advantageously enable successful completion of minimally invasive procedure in more difficult cases. In tortuous vasculature, for example, when a calcified blockage is encountered in which a stent is to be deployed (e.g., as disclosed in U.S. Provisional Application Ser. No. 63/655,044, incorporated herein by reference), the transition from the dilator to the proximal end of the GEC may determine the success of the procedure. Fishmouthing at the junction between the dilator and GEC, may, for example, cause the GEC to snag and fail to follow the dilator through the blockage. A smooth ‘snug’ fit may, for example, advantageously enable the GEC to successfully follow the dilator through the blockage. Accordingly, the LFD may advantageously reduce the need for major (e.g., open cavity) surgery and/or other major (e.g., more costly, more life-threatening) procedures.

The dilator 120 includes a body 125 with a flexible tip 130. The tip 130 includes a tapered end 135. The tapered end 135 may be configured for advancement through the vasculature 100. This configuration may advantageously provide atraumatic navigation through tortuous anatomy. An axial translation member 140 in the form of a push rod, in the depicted example, is coupled to an axial translation module 145 in the form of a hypo tube push member. The axial translation module 145 is configured to receive pushing force from the axial translation member 140. The pushing force may be transmitted through the axial translation module 145 to advance the dilator 120. This arrangement may, for example, advantageously enable precise control of dilator advancement while maintaining a low profile configuration.

A smooth, fitted transition from dilator to GEC may, for example, advantageously enhance tracking performance through complex vascular anatomies. A fitted interface between the dilator and GEC may, for example, advantageously reduce risk of vessel wall trauma, such as by eliminating irregular contact points, by way of example and not limitation. The maintained coaxial alignment between the dilator and GEC may, for example, advantageously improve precision during interventional procedures. Additional advantages may, for example, include reduced friction during catheter manipulation, improved subsequent device deployment, and/or enhanced overall procedural efficiency and reliability. One or more of these illustrative advantages may, by way of example and not limitation, be achieved through one or more of the example LFD embodiments disclosed herein.

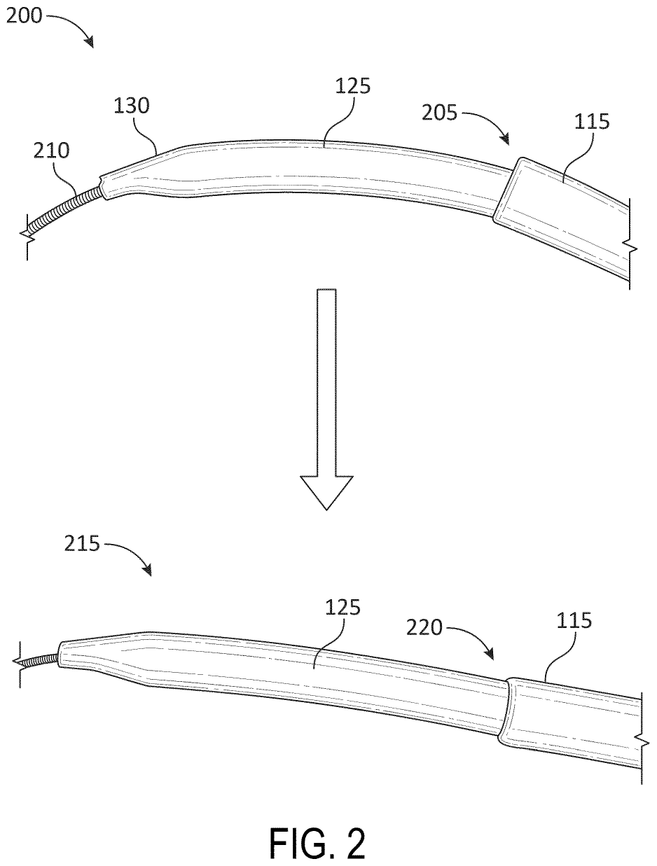

FIG. 2 depicts an example LFD contrasted to a traditional dilator with a gaping mouth formed by a loose fit between the GEC lumen and the outer diameter (OD) of the dilator 120. As shown, a dilator body 125 in a traditional configuration 200 results in a gaping mouth 205 between the dilator and GEC 115. In this example, a guide wire 210 is shown disposed through an inner lumen of the dilator 120. In contrast, in a lumen-fitted configuration 215, a dilator 120 maintains a fitted transition 220 from the dilator 120 to the GEC. This fitted transition may, for example, advantageously increase passability of the GEC 115, reduce tissue damage, and/or improve success rates (e.g., through tortuous anatomy).

FIG. 3 depicts the example LFD maintaining a smooth fitted transition 220 from dilator 120 to GEC in an illustrative flexed configuration, such as the GEC-LFD assembly may be operated into while traversing tortuous passages. For example, as the assembly is flexed (such as through multiple complex bends in peripheral and/or coronary vasculature while approaching a calcified obstruction), the fitted transition 220 may advantageously enable the GEC to be passed into and/or through the obstruction following the path of the dilator without ‘snagging’ and folding back on itself against an edge of the obstruction.

FIG. 4 depicts an illustrative LFD assembled within an illustrative dilator-fitting GEC (DFGEC). The GEC 115 includes a contoured distal end 405 configured to flexibly and slidably engage the outer diameter of the dilator 120 (an LFD, as depicted). In the depicted example, the contoured distal end 405 is contoured (e.g., tapered) down to substantially match the outer diameter of the LFD. The fitted transition 220 between the LFD (dilator 120) and GEC 115 may, for example, advantageously be maintained while allowing relative axial and rotational movement between the LFD and GEC 115. This arrangement may, for example, advantageously provide smooth traversal through vasculature while maintaining freedom of movement between the dilator 120 and GEC 115 during navigation of tortuous anatomy.

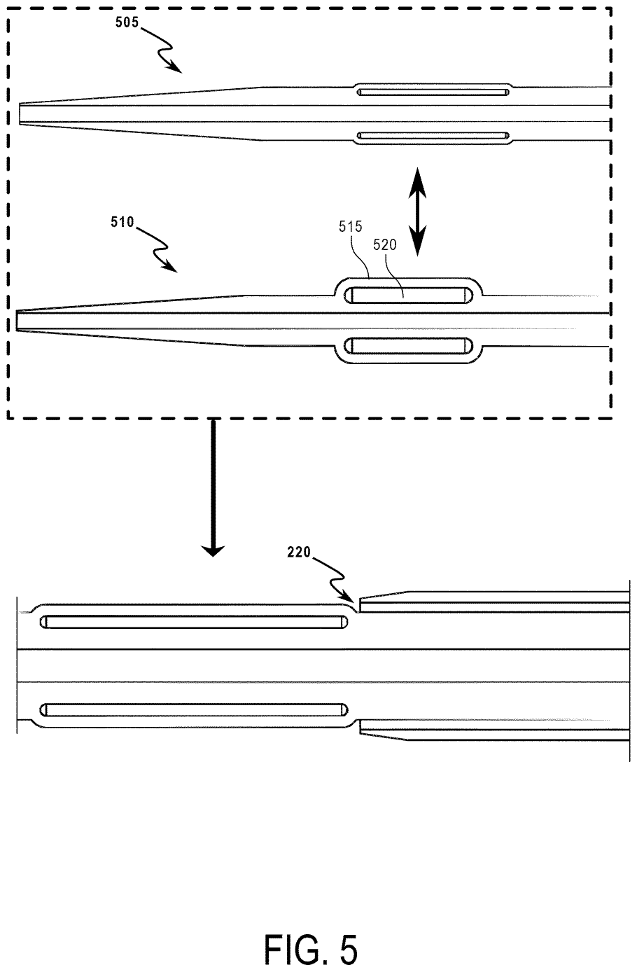

FIG. 5 depicts an illustrative LFD with adjustable lumen fitment. A passage mode 505 of the LFD includes a selective lumen fitment module 515 in a collapsed configuration. For example, the LFD (e.g., including the selective lumen fitment module 515) may pass through the lumen of the GEC in the passage mode. The fitment module may, for example, be operated into an expanded configuration. An occlusion mode 510 shows the LFD with the selective lumen fitment module 515 in an example expanded configuration. In this example, the selective lumen fitment module 515 includes a bladder 520 configured to expand (e.g., selectively such as by an operator) to form a fitted transition 220 to the lumen of the GEC 115. This configuration may, for example, advantageously prevent fish-mouthing while maintaining a low profile.

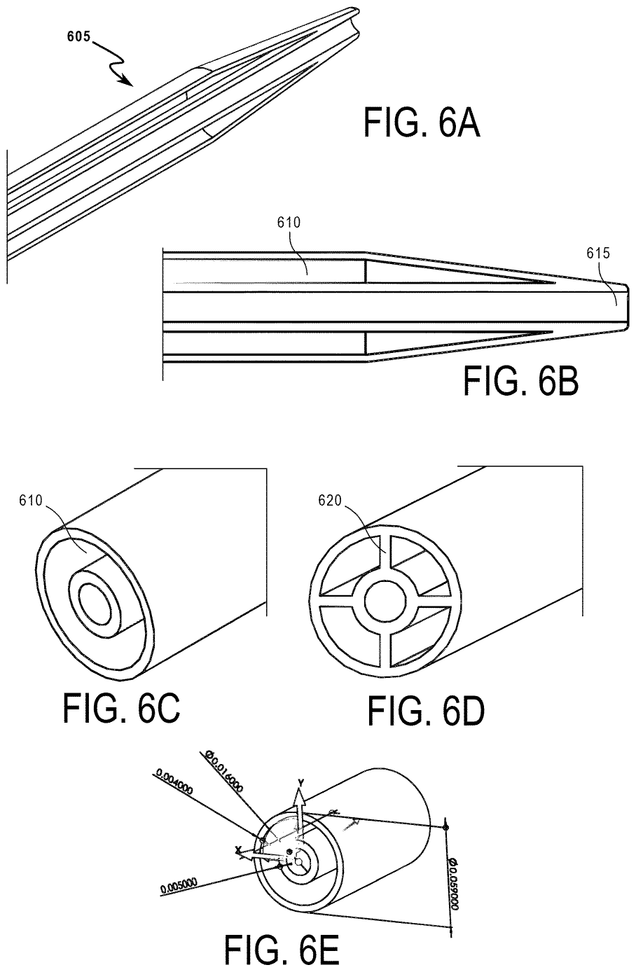

FIG. 6A and FIG. 6B depict cross-section views along a longitudinal axis of an illustrative self-adjusting outer diameter LFD (SAODLFD) 605. The SAODLFD 605 includes a bladder 610 disposed between outer and inner walls. The bladder 610 may, for example, be hollow such as depicted. The bladder 610 may, for example, be at least partially filled with a fluid and/or solid material. An inner lumen 615 extends through the LFD. The inner lumen 615 may, for example, be configured for passage of fluid and/or other substance (e.g., a guide wire). SODLFD configurations, such as the example depicted, may, for example, advantageously allow the outer diameter to self-adjust while maintaining a consistent inner lumen diameter.

FIG. 6C is a cross-section view of a SAODLFD across the longitudinal axis. As shown, support elements 620 are disposed within the bladder 610. The support elements 620 may provide structural support while allowing controlled deformation of the bladder 610. This arrangement may advantageously prevent collapse of the inner lumen while enabling outer diameter adjustment.

FIG. 6D is a cross-section view showing support elements configured as longitudinally-extending ribs. The ribbed configuration may advantageously provide, for example, directional support while maintaining flexibility. FIG. 6E is a cross-section view showing illustrative dimensions. The dimensions may, for example, be selected to optimize the balance between outer diameter adjustability and inner lumen patency.

FIG. 7A depicts an illustrative LFD with a selectively-actuated adjustable lumen fitment module 705. The module 705 may, for example, be configured to selectively expand in diameter. An example expansion module 710 may include, such as depicted, a braid configured to maintain a predetermined volumetric relationship. A longitudinal actuation member 715 in the form of a sleeve, in this example, longitudinally compresses the radially expanding module. Motion M7A indicates radial expansion and/or contraction. The radial adjustment may, for example, advantageously provide selective fitment to the GEC inner diameter. Motion M7B indicates longitudinal displacement. The longitudinal displacement (e.g., induced by the longitudinal actuation member 715), may, for example, induce actuation of the longitudinal actuation member. Selectively actuated adjustable lumen fitment configurations may, for example, advantageously provide controlled expansion while maintaining structural integrity.

FIG. 7B depicts an example LFD with a lumen fitment module configured as a hub sized and positioned to limit axial advancement of the LFD through a GEC. FIG. 7C depicts a cross-section view along a longitudinal axis of the assembled LFD with lumen fitment module of FIG. 7B in an illustrative use case in which the LFD is inserted into a GEC. A distal portion 720 of the LFD body includes a lumen fitment module 725 configured as an expanded ‘bell’ end. A proximal portion 730 extends proximally from the lumen fitment module 725. In the depicted example, the lumen fitment module 725 may, for example, limit advancement of the dilator through the GEC 115 (e.g., to the left in FIG. 7C). For example, the GEC 115 may be configured as a DFGEC. Such embodiments may, for example, advantageously prevent over-advancement of the dilator and/or may, for example, advantageously enable the dilator to act as a ‘push’ member for the GEC. For example, the proximal portion 730 may be configured on a proximal end as an axial advancement member (e.g., as disclosed at least with reference to FIGS. 14-21).

In some embodiments, a hub may, for example, be provided at a distal end of the GEC. In some examples, the hub may, for example, be compressible. For example, the hub may compress to fit inside the GEC and expand when passed through the GEC. This arrangement may advantageously provide a smooth transition while preventing fish-mouthing.

FIG. 8 depicts an illustrative longitudinally-relieved LFD. A longitudinally-extending relief 805 allows expanded outer diameter of the dilator by allowing the relief to fit over an obstruction in the lumen such as a push rod weldment. This configuration may advantageously accommodate internal components while maintaining outer diameter control.

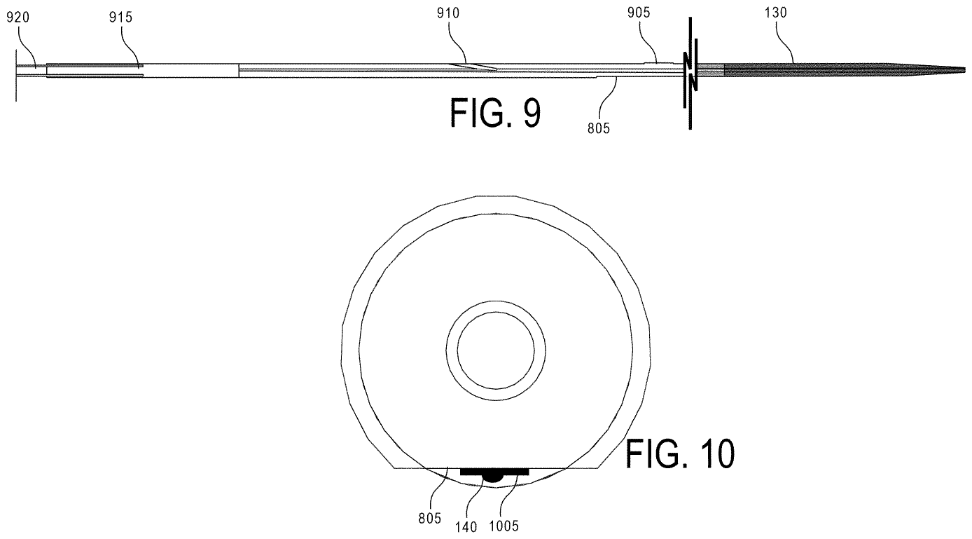

FIG. 9 depicts an illustrative cross-section view along the longitudinal axis of the longitudinally-relieved LFD, in an embodiment including a lumen access port and a circumferential axial translation module. A collar 905 provides an expanded fit within the inner lumen of the GEC during obstruction engagement operations. A lumen access port 910 provides access to the inner lumen of the LFD from a circumferential exterior. A circumferential engagement translation module (CETM) 915 is configured to axially advance the dilator by engaging at least a portion of a circumference of the dilator. A dilator tail 920 extends proximally. This configuration may advantageously provide multiple functional features while maintaining a streamlined profile.

FIG. 10 depicts a view from the distal end towards the proximal end across the longitudinal axis of the LFD depicted in FIGS. 8-9. A weldment 1005 protrudes radially outward from the push rod (axial translation member 140). The relief 805 accommodates this protrusion while maintaining overall diameter control.

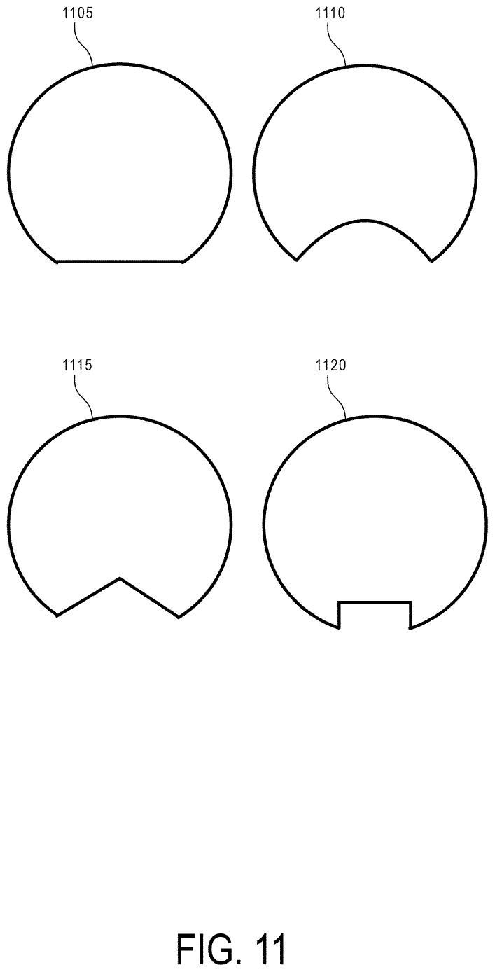

FIG. 11 depicts illustrative cross-sectional geometry across the longitudinal axis of example LFDs. A D-shaped cross-sectional geometry 1105 includes a flat relief, such as shown in FIGS. 8-10. A cross-sectional geometry 1110 includes a curved relief. A cross-sectional geometry 1115 includes a polygonal relief. A cross-sectional geometry 1120 includes a notched (e.g., polygonal as shown) relief. These various geometries may advantageously provide different mechanical properties and accommodation of internal components.

FIG. 12 depicts an LFD operably coupled to a separate CETM. An axial interdigitation module 1205 of the CETM 915 engages with an axial interdigitation module 1210 of the LFD. The LFD body 125 including the axial interdigitation module 1210 is shown partially transparent in the close-up view. Interdigitated engagement may, for example, advantageously reduce clearance requirements between the outer surface of the dilator and the inner lumen of the GEC such as, for example, by permitting circumferential engagement by a push member instead of, by way of example and not limitation, bonding to a push rod laminated on the outside surface of the dilator.

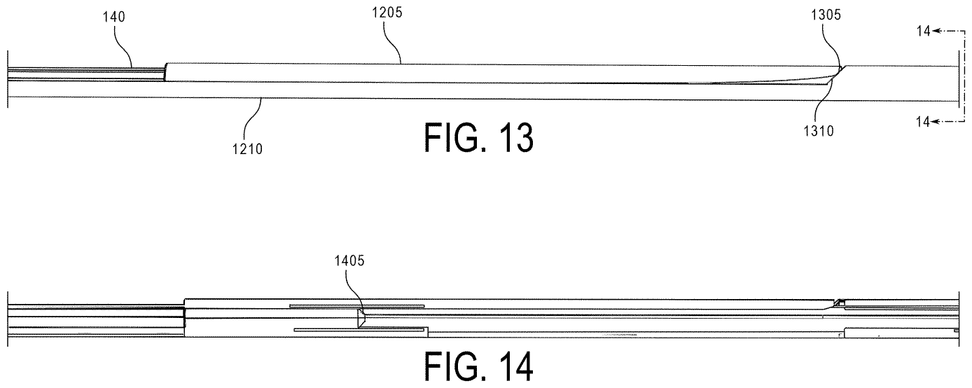

FIG. 13 depicts a DFGEC with integrated axial interdigitation module operably engaging an axial interdigitation module of an LFD. An LFD engagement tip 1305 of the axial interdigitation module 1205 of the CETM 915 interfaces with an engagement face 1310 of the axial interdigitation module 1210 of the LFD. The DFGEC may, for example, advantageously reduce clearance requirements, such as disclosed at least with reference to FIG. 12. An interdigitating interface may, for example, advantageously provide positive engagement while maintaining alignment. In some embodiments, by way of example and not limitation, the interdigitating surfaces (e.g., 1205, 1210, 1305, 1310) may, for example, be formed directly into the tube material (e.g., by laser-cutting, molding).

FIG. 14 depicts a cross-section view along the longitudinal axis of the view of FIG. 13. An aperture 1405 into the lumen of the LFD includes a tapered inner diameter configured to guide a guide wire into the inner lumen of the LFD. This tapered configuration may advantageously facilitate smooth wire introduction while maintaining proper alignment.

FIG. 15 depicts an illustrative LFD with a stepped-diameter distal tip and an example DFGEC with integrated axial interdigitation module. An outer diameter step 1505 transitions from the body 125 of dilator 120 to the dilator tip 130. This stepped configuration may, for example, advantageously provide improved trackability while maintaining proper fit.

FIG. 16 depicts an illustrative CETM which may be integrated with the GEC body. The CETM 915 may be coupled to or formed as part of a GEC and/or LFD (e.g., on a proximal end). The axial translation member 140 may be integrated into the CETM 915. The CETM may be formed (e.g., laser-cut, heat-formed, stamped) to provide various features. This integrated configuration may advantageously reduce parts while maintaining functionality.

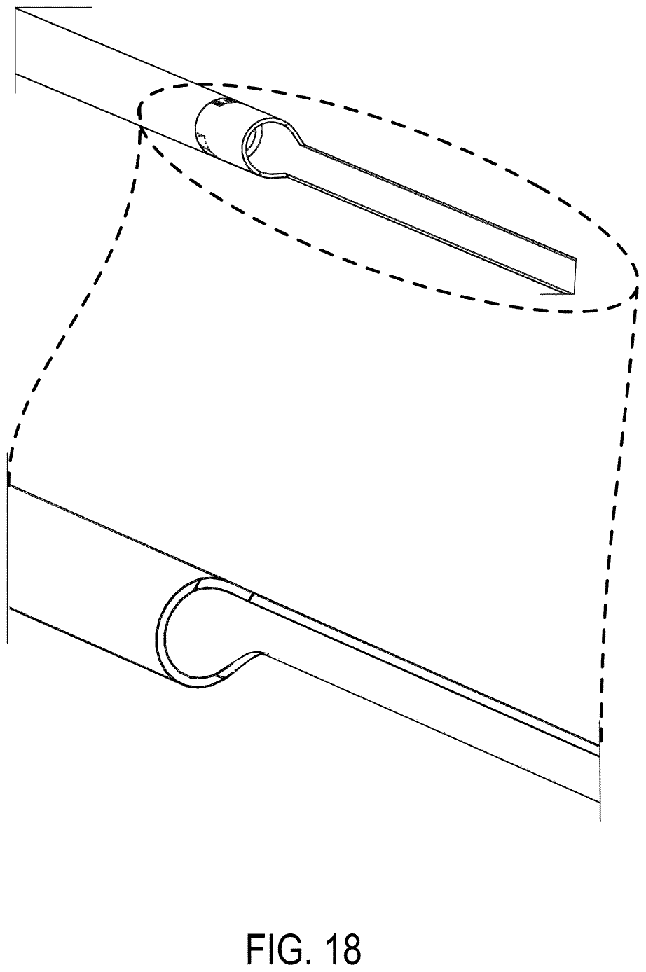

FIG. 17 depicts an illustrative CETM which may be coupled to the proximal end of the GEC. FIG. 18 depicts an example CETM coupled to the proximal end of the GEC. The CETM may, for example, be coupled to the proximal end of the GEC (and/or an LFD). For example, the CETM may be coupled to a tube via press-fit and/or snap-fit. The CETM may, for example, be adhered to a tube. The CETM may, for example, be welded to the tube (e.g., sonic welding, thermal welding, solvent welding). The CETM may, for example, be over-molded on the tube and/or the tube over-molded onto the tube. The CETM may, for example, be laminated to the tube (e.g., or vice versa). The CETM may, for example, be unitarily formed (e.g., of continuous material) with the tube. The CETM 915 includes an integrated axial translation member. A modular CETM configuration may, for example, advantageously enable customization and/or variety in sourcing.

FIG. 19 depicts an example CETM configured to receive an illustrative planar-assembled axial translation member. A mating feature 1905 in the form of a notch in CETM 915 is configured to receive an axial translation member 140 substantially in plane with the CETM 915. This planar configuration may advantageously provide a low profile while maintaining proper alignment. For example, by coupling the axial translation member 140 in plane to the CETM, a clearance requirement between the dilator and GEC may, for example, be reduced (e.g., by moving the weld such as disclosed at least with reference to FIG. 10). In some embodiments, the CETM may, for example, be formed into the GEC (e.g., as disclosed at least with reference to Appendices D-E).

FIG. 20 depicts a reduced-clearance DFGEC assembly including the CETM of FIG. 19 assembled substantially in-plane with an axial translation member. A mating region 2005 joins the axial translation member 140 to the mating feature 1905. The axial translation member 140 may be welded to the mating feature 1905. The reduced-clearance joint formed at the mating region 2005 permits a larger effective outer diameter of dilator to be used in the resulting GEC without binding the dilator in the GEC. The increased outer diameter of the LFD, for example, may advantageously provide a smoother transition between the GEC and the LFD and increase ‘pushing’ability of the dilator into and through a lesion.

FIG. 21 depicts a dilator (e.g., LFD) with a lumen access port (e.g., such as the lumen access port shown in FIG. 9). The lumen access port 910 provides access to the inner lumen of the LFD from a circumferential exterior of the LFD. The lumen access port 910 may be configured, for example, to allow fluid communication between the inner lumen and an exterior environment. A lumen access port(s) configuration may, for example, advantageously enable introduction or withdrawal of fluids while maintaining the dilator position.

FIG. 22 depicts a method 2200 of deploying an embedded device (e.g., a stent) in the body (e.g., in tortuous vasculature). The method 2200 includes operating a medical device (e.g., stent as depicted, graft) inside a tube in a step 2205. The tube may, for example, include a lumen of a dilator (e.g., inner lumen 615). The tube may, for example, include a microcatheter (e.g., without a dilator, disposed inside a dilator). The tube may, by way of example and not limitation, have an inner diameter just sufficient for a collapsed stent (e.g., 0.025 inches or less).

The medical device may, for example, be advanced in a collapsed configuration within the tube. The method 2200 includes advancing a dilator into a lesion in a step 2210. If the dilator is not sufficiently in the lesion (decision point 2215), the method returns to step 2210. When the dilator is determined to be sufficiently in the lesion (e.g., measured by displacement, tactile ‘pops,’ external imaging), the method includes advancing the tube into the lesion in a step 2220. Once it is determined in a decision point 2225 that the medical device is within the lesion, then the tube is retracted in a step 2230, leaving the medical device behind. The medical device is operated into a deployment mode in a step 2235.

For example, in some embodiments a medical device may be disposed over a central tube. The medical device may, for example, be expandable. The medical device may, for example, be expanded by forcing a substance (e.g., fluid, solid) within the lumen. The region of the lumen within the stent may, for example, be expandable (e.g., sock, balloon). Accordingly, forcing the substance within the lumen may induce expansion of the stent.

In some embodiments, the medical device may, for example, be self-expanding. A tube may, for example, be forced over the medical device, (e.g., operating or after operating the medical device into a collapsed configuration). When the medical device is translated out of the tube, it may self-expand.

In some embodiments, the tube may be actively expandable. For example, a portion of the tube configured to be operated into the lesion may include a shape memory alloy and/or polymer (e.g., nitinol). The portion of the tube may, for example, be actively operated to expand.

In some embodiments a dilator may, for example, be omitted. Steps 2210-2215 may include advancing the tube (e.g., microcatheter) into the lesion. Step 2220 may, for example, be omitted.

In some embodiments, the tube may, for example, be the dilator. Step 2220 may, for example, be omitted.

FIG. 23A, FIG. 23B, and FIG. 24 depict illustrative fenestrated guide extension catheters (GECs). FIG. 23A depicts a fenestrated GEC assembly 2305. In this example, the fenestrated GEC assembly 2305 includes a GEC 115. A dilator 120 is disposed within the GEC 115. The dilator 120 is depicted as extending from the distal end of the GEC 115. The fenestrated GEC assembly 2305 includes a fenestration region 2310, which is shown along a longitudinal portion of the GEC 115. The fenestration region 2310 includes multiple apertures 2315 distributed along its length and/or around its circumference. These apertures 2315 may, for example, provide communication (e.g., fluid communication) between the interior and exterior of the GEC 115. Communication between the lumen of the GEC 115 and a surrounding environment may, by way of example and not limitation, provide fluid transfer and/or pressure equalization. In various embodiments, the fenestrations in the wall of the GEC 115 may, for example, advantageously allow blood flow from a larger proximal vessel into the lumen, promoting perfusion through the distal end.

In the depicted example, the GEC 115 includes a distal region 2320 distal to the fenestration region 2310. As shown, in some embodiments the distal region 2320 may be solid (e.g., non-fenestrated). The distal region 2320 may be configured (e.g., sized) according to a specific target function. For example, the length may be configured according to a device to be delivered (e.g., according to a length of a stent). The length may, for example, be configured according to a target embedment (e.g., a length of intended deep-seating, such as in a calcified lesion).

FIG. 23B depicts an embodiment of a fenestrated GEC assembly 2305 with an extended distal region 2320. The extended distal region 2320 may, by way of example and not limitation, advantageously sheath a longer stent and/or seat deeper in a calcified lesion than an embodiment with a shorter distal region 2320, such as shown in FIG. 23A.

In some embodiments, such as shown in FIGS. 23A-23B, the distal region may have a length greater than 10 mm. For example, the distal region may be longer than 30 mm. In some embodiments, the distal region may be longer than 40 mm. The distal region may be longer than 50 mm, for example. The distal region may, for example, be at least 10 cm. In some embodiments (e.g., for stent placement), the distal region 2320 may be between 10-15 cm long. The distal region 2320 may, for example, be greater than 15 cm long. In some examples, such as shown in FIG. 24, the distal region 2320 may be shortened (e.g., or omitted). Embodiments with shorter distal regions may, for example, advantageously enable delivery of fluid closer to a distal tip of the GEC 115.

Some embodiments, such as those depicted in FIGS. 23A-24, may have more than 2 apertures. For example, some embodiments may have more than 4 apertures. Some embodiments may have a 5, 10, or more apertures. For example, in the event of distal region occlusion, such configurations may avoid vessel perforation and/or dissection if elevated pressure is applied (e.g., fluid is injected) through the lumen and escapes out the fenestration region 2310.

In some embodiments, the total aperture area and/or longitudinal distribution may vary, with examples showing differentiation along the structure's length. Variations in aperture number and/or size, (e.g., changing with respect to position along the longitudinal axis) may be provided.

FIG. 24 depicts a fenestrated GEC assembly 2305 provided with an occlusion module 2405 at the distal region 2320. The occlusion module 2405 may, for example, be operable to selectively block one or more apertures 2315 located within the fenestration region 2310 along the GEC 115. Selective occlusion of the apertures 2315 may, for example, be achieved by adjusting the position of the occlusion module 2405 relative to the apertures 2315. In this example, the left side of the image shows the occlusion module 2405 operated into a blocking position. Axial advancement of the dilator 120 (as shown by extension motion M24A) operates the occlusion module 2405 into a non-blocking mode. Embodiments configured to selectively occlude the apertures 2315 may, for example, advantageously enable controlled fluid communication between the interior and exterior of the GEC. Such embodiments may, for example, advantageously facilitate pressure management and/or isolation of specific vascular regions during a medical procedure within the vasculature.

In some examples, embodiments with fenestrated regions (e.g., with more than 2, 3, or 4 apertures, for example, such as more than 5 apertures, more than 10 apertures) may, for example, provide advantages beyond mere systemic pressure monitoring. For example, a GEC and/or dilator with a hydrophilic coating may experience adhesion (e.g., intravascular adhesion) under compression conditions (e.g., when ‘stuck’ for an extended period of time in a lesion without axial translation). A fenestration region may, for example, advantageously enable re-wetting such as, for example, by releasing fluid from the lumen proximally to the adhesed region.

In some embodiments, for example, an expansion module of a dilator 120, such as shown in FIGS. 26A-B, may be operated proximally of a fenestration region 2310, as depicted in FIG. 24. The expansion module may, for example, be configured to expand the outer radial circumference of a fenestrated GEC assembly 2305. Positioned proximally of the fenestration region 2310, the expansion module may, for example, encourage the fluid to flow towards the distal region 2320. The distal flow may, for example, be facilitated by the obstruction created by the expansion module, which redirects the released fluid towards the distal end of the GEC 115. This arrangement may, for example, advantageously enhance localized delivery of fluid to targeted vascular regions, thereby improving the efficacy of procedures conducted using the fenestrated GEC assembly 2305.

Fenestrated embodiments may advantageously enable, for example, injection of medications. Example medications may, for example, include nitric oxide-related agents (e.g., sodium nitroprusside, nitric oxide). Example medications may, for example, include thrombolytics and/or other platelet inhibitors (e.g., glycoprotein 2b3a inhibitors, P2Y12 inhibitors) such as, for example, may advantageously deter active thrombus formation. Example medications may, for example, include adenosine. Such embodiments may, for example, advantageously facilitate rapid correction in no-reflow situations, such as microvascular failure, e.g., without requiring removal of the GEC (e.g. for insertion of a smaller micro-aspiration catheter).

FIG. 25 depicts an illustrative lumen-fitted dilator (LFD) provided with a lumen-dilating module in an illustrative lumen-expanding dilator GEC assembly 2505. An example lumen-expanding module 2515 is located proximate to the distal end of the dilator 120. The lumen-expanding module 2515 may be configured to selectively dilate the GEC 115. In this example, the lumen-expanding module 2515 is configured to selectively radially expand a radial expansion region 2510 of the GEC 115. Lumen-expanding embodiments may, by way of example and not limitation, form a fitted transition between the dilator 120 and GEC 115, which may, for example, advantageously reduce the likelihood of fish-mouthing during medical procedures.

The lumen-expanding module 2515 may, for example, be configured to maintain a smooth profile during insertion while allowing controlled lateral expansion. Controlled expansion may, for example, increase lumen patency and/or reduce trauma as the LFD traverses a tortuous anatomical region and/or enters a restricted region (e.g., a lesion).

The module 2515 may, for example, be configured as an enlarged (e.g., permanently, selectively) region of the dilator 120. The enlarged region may, for example, be retracted proximally to the radial expansion region 2510. For example, in the radially collapsed mode, the GEC 115 may be advanced into a lesion. As the dilator 120 is advanced distally, the lumen-expanding module 2515 the radial expansion region 2510 may stretch to permit the dilator 120 to advance. In some embodiments, for example, the dilator may be stepped (e.g., such as disclosed at least with reference to FIG. 15).

In an advancement configuration (e.g., when advancing in the vasculature), a distal tip of the radial expansion region 2510 may be collinear with the tip of the dilator 120 (e.g., the distal tip, or just proximal to the tapered tip). For example, the radial expansion region 2510 may conform to the dilator 120.

FIG. 26A depicts a lumen-expanding module 2515 positioned close to the distal tip of the dilator 120. This proximity configuration may be advantageously aligned with applications requiring a short stent deployment. The near-tip position of the lumen-expanding module 2515 may, for example, facilitate rapid expansion at or near an obstruction, providing localized dilation focused at the distal end of the guide extension catheter 115.

FIG. 26B illustrates an embodiment in which the lumen-expanding module 2515 is situated farther away from the distal tip of the dilator 120. This extended position may be particularly suited for operations involving a longer stent. The increased distance between the module 2515 and the dilator tip may, for example, advantageously accommodate a longer stent. Such embodiments may, for example, advantageously allow for a gradual, controlled dilation that smaller or more proximal expansion configurations may not support. By positioning the module 2515 further from the tip, the dilator may, for example, maintain low-profile passage initially, enabling a more uniform expansion over an elongated segment of the catheter assembly, which may improve the expansion and/or stabilization of deeper engagement in a restricted region (e.g., a lesion).

In some embodiments the radial expansion region 2510 may, for example, have a smaller cross-sectional area (e.g., smaller diameter) across the longitudinal axis than the proximal remainder of the GEC 115. A smaller region 2510 may, for example, advantageously conform to the distal end of the dilator 120 (e.g., a smaller cross-sectional area across the longitudinal axis of the dilator 120).

The radial expansion region 2510 may, for example, include a polymer with a low friction coefficient (e.g., at the molecular level) such as PTFE (polytetrafluoroethylene), PEEK (polyetheretherketone), and/or HMWHDPE (high molecular weight (HMW) high density polyethylene (HDPE)). Such polymers may, for example, advantageously provide high lubricity of the outside surface of the GEC. The radial expansion region 2510 may, for example, include a polymer (e.g., in combination with PTFE) with a lower elastic modulus than PTFE. The lower elastic modulus may, for example, advantageously contribute a ‘stretchy’ characteristic to the radial expansion region 2510.

A guide extension catheter (GEC) may, for example, have a wall thickness of approximately 0.005 inches. A wall thickness may be, for example, 0.0045 inches. A wall thickness may, for example, be 0.0040 inches. A wall thickness may, for example, be 0.0035 inches. A wall thickness may, for example, be 0.0030 inches. A wall thickness may, for example, be 0.0025 inches. A wall thickness may, for example, be 0.0020 inches. A wall thickness may, for example, be 0.0015 inches.

Thin wall designs, such as described above, may, for example, advantageously offer increased flexibility (e.g., of the radial expansion region 2510), which may be beneficial during advancement through the vasculature. Thin walled regions may, for example, be radially supported (e.g., from collapsing) by the dilator 120.

In some scenarios, the thin and flexible nature of the radial expansion region 2510 may prevent the dilator from being retracted once displaced. The assembly may, for example, be configured to allow the dilator 120 to be pushed through the GEC 115 (e.g., to facilitate stent deployment). The dilator 120 may, for example, be withdrawn and/or replaced with a smaller and/or non-expanding dilator.

In some embodiments, a dilator 120 may be a single entity with a module (e.g., the lumen-expanding module 2515), such as being unitarily formed (e.g., from a single composition such as during molding). In some embodiments, the dilator and module (e.g., lumen-expanding module 2515) may, for example, be assembled from multiple pieces (e.g., two or more). For example, some embodiments may be field assembled. A physician may, for example, select a lumen-expanding module 2515 for use with a specific dilator 120 and/or GEC 115.

Some embodiments may include stacked and/or staged dilator features. Stacked and/or staged dilator features may include serial expansion modules and/or serially stepped cross-sectional areas. Such embodiments may, for example, enable stepped dilation of vascular lesions.

In some embodiments, an example implementation of a GEC may include a 6 French (Fr) GEC lumen distally transitioning to a 5 Fr lumen. A standard stent may, for example, just fit within a 6 Fr lumen. Within some implementations, the primary length of the GEC may have a 6 Fr dimension, while a distal end region (e.g., the radial expansion region 2510) may be smaller, such as 5 Fr. The distal end region may, for example, expand to the primary or larger diameters, such as to 6 Fr or greater. Such designs may, by way of example and not limitation, advantageously enable flexibility in use and/or accommodate varied application requirements.

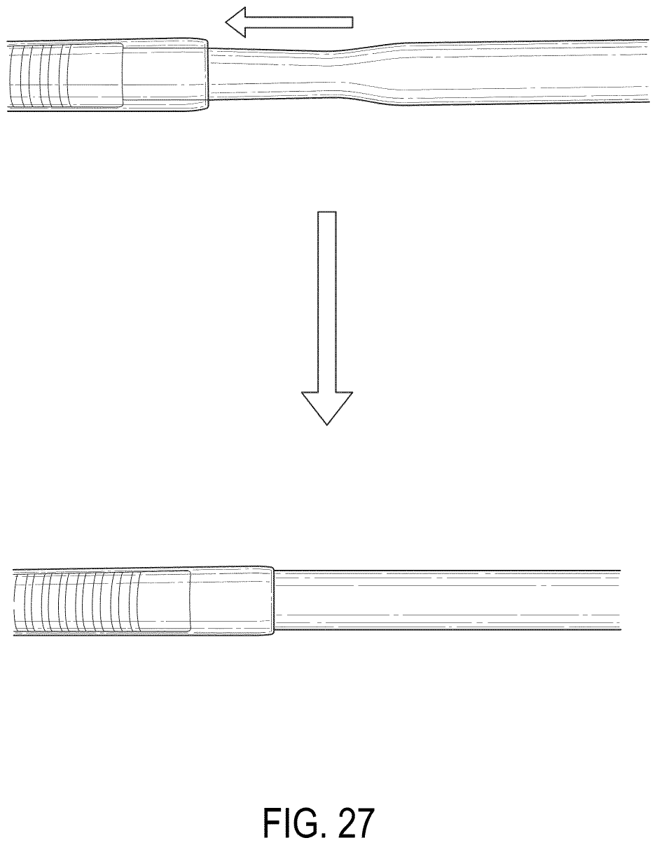

FIG. 27 depicts an illustrative LFD being inserted into a GEC. The LFD includes, for example, a stepped region with a change in cross-sectional area across the longitudinal axis. As the step is brought into alignment with the mouth (aperture into the lumen) of the distal end of the GEC, a fitted transition is formed between the LFD and the GEC.

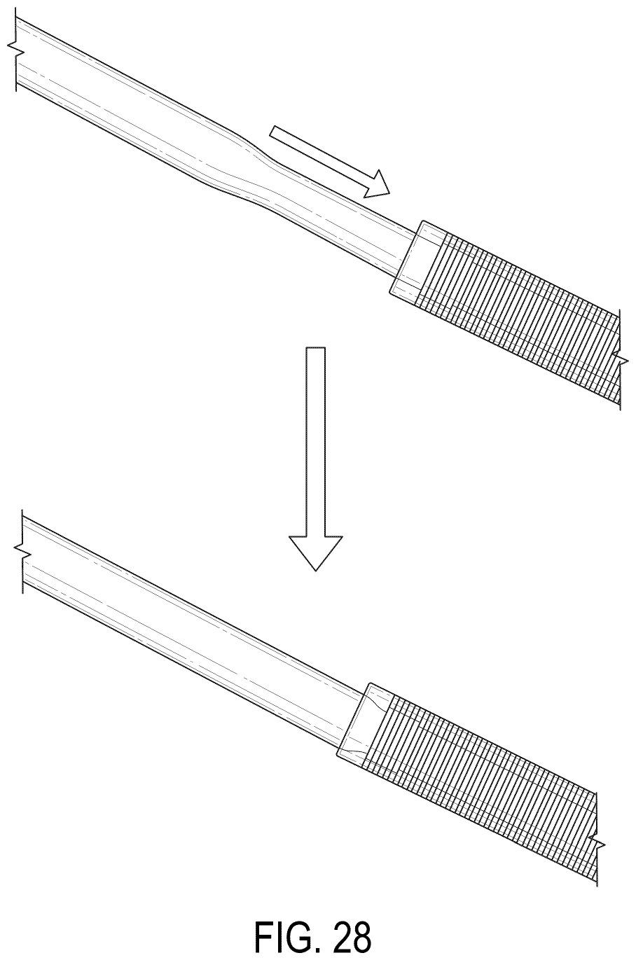

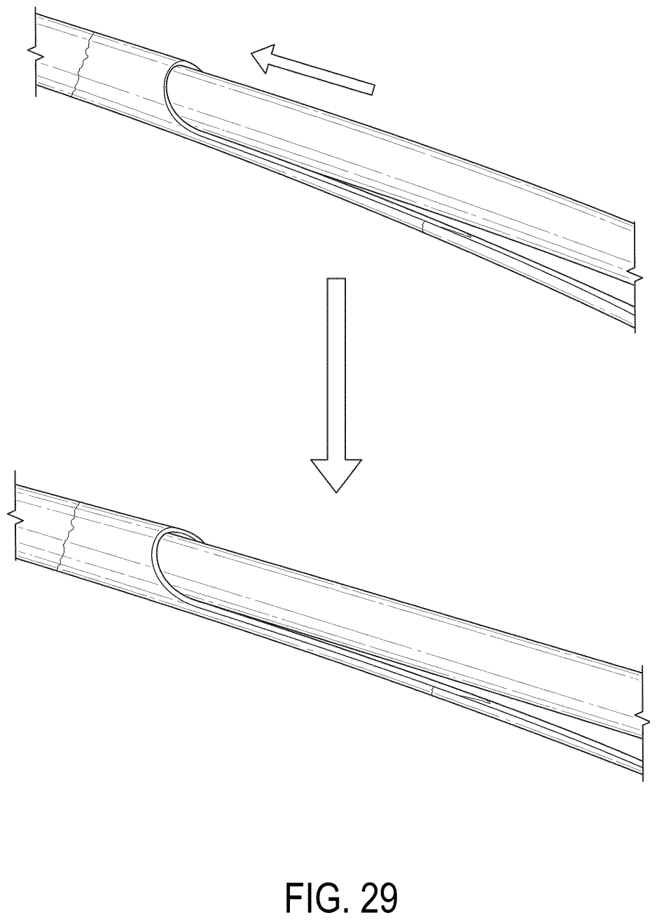

FIG. 28 depicts an illustrative LFD being inserted into a clear GEC (reinforced with an embedded coil, as depicted). The LFD includes, for example, a stepped region with a change in cross-sectional area across the longitudinal axis. As the step is brought into alignment with the mouth (aperture into the lumen) of the distal end of the GEC, a fitted transition is formed between the LFD and the GEC.

FIG. 29 depicts an illustrative embodiment of an LFD with axial engagement module. The LFD includes an integrally molded axial engagement module. A corresponding axial engagement module is inserted and ‘wedged’into an opening of the axial engagement module of the LFD.

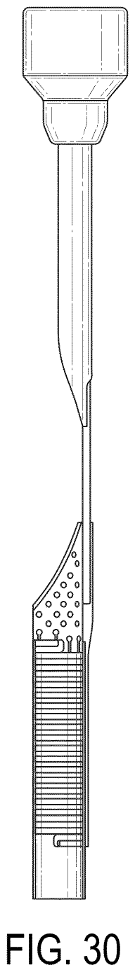

FIG. 30 depicts an illustrative implementation (shortened for ease of viewing) of a DFGEC.

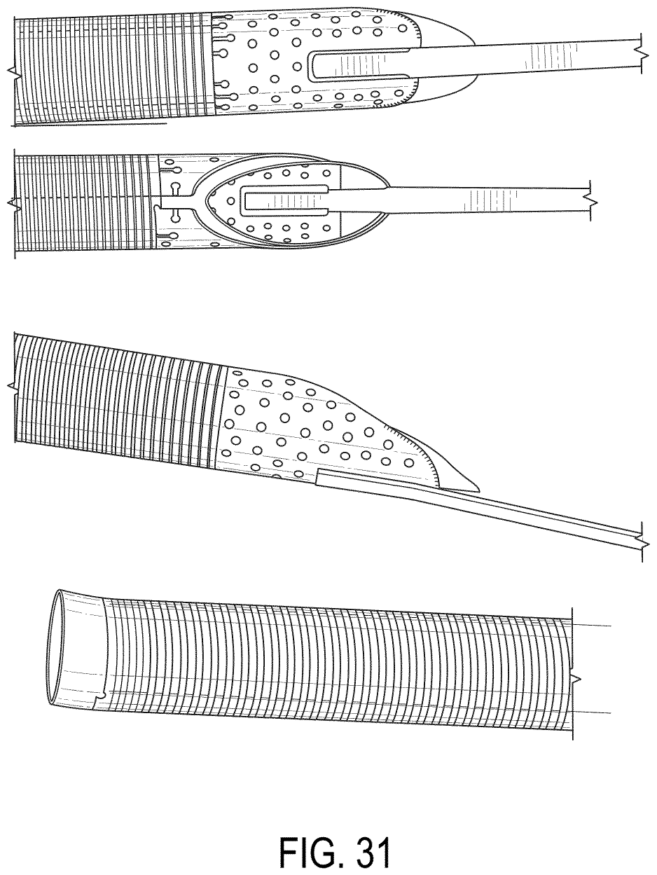

FIG. 31 depicts illustrative views of the embodiment depicted in FIG. 30. As shown, an axial translation member is fitted into (e.g., flush) a corresponding cavity (e.g., notch, as shown) in the DFGEC. The depicted weldment between the axial translation member and the DFGEC may, for example, be (substantially) flush with the inner and/or outer surface of the DFGEC. The fitted joining of the axial translation member may, for example, reduce or avoid interference with fitment of the DFGEC into a lumen and/or fitment of an object through the lumen of the DFGEC.

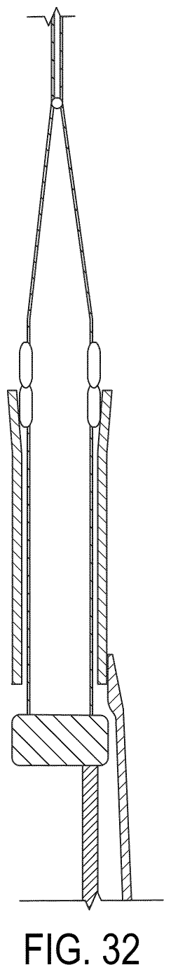

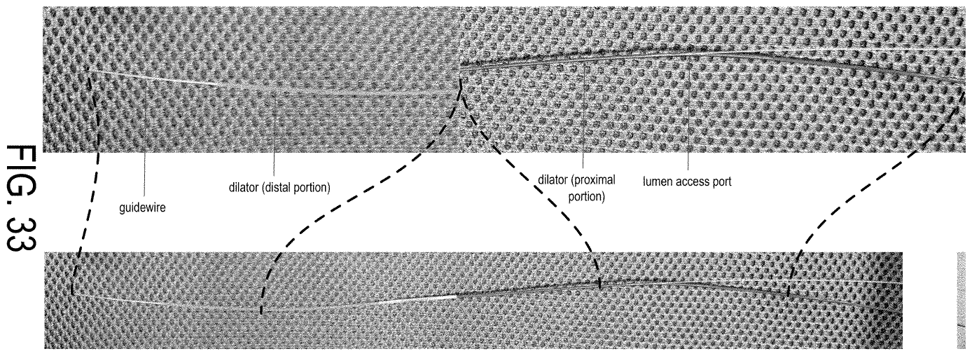

FIG. 32 depicts an illustrative implementation (cross-section view along the longitudinal axis, and shortened for ease of depiction) of a DFGEC and GEC-matched LFD (e.g., such as disclosed at least with reference to FIG. 5, FIGS. 7B-7C).

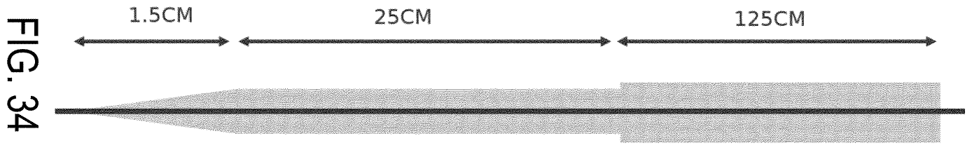

FIG. 33 depicts an illustrative prototype of an LFD with a lumen access port, such as disclosed at least with reference to FIGS. 9 and 22.

FIG. 34 depicts an illustrative proportional relationship of a dilator (left two portions) and GEC (right portion).

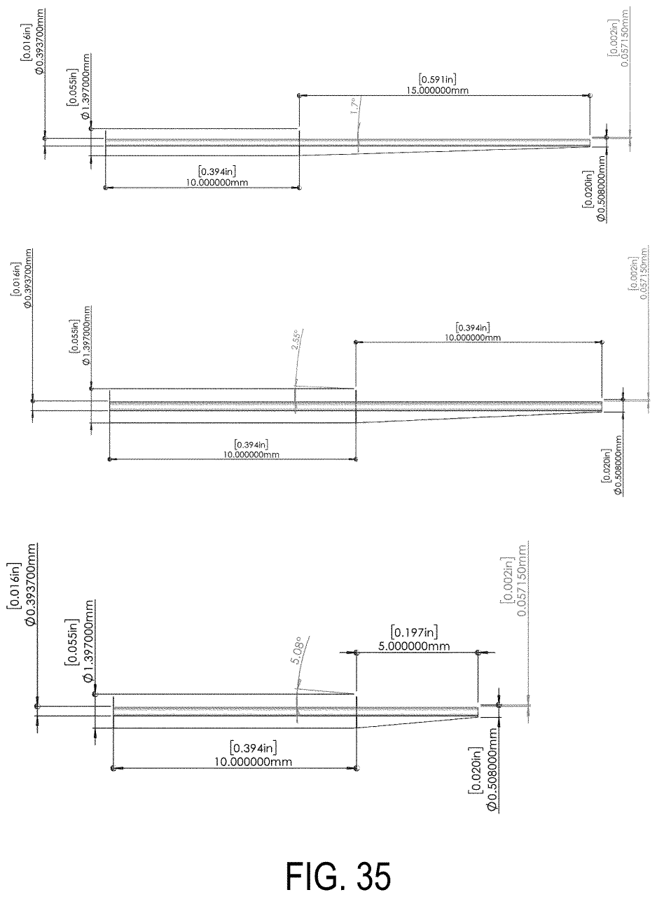

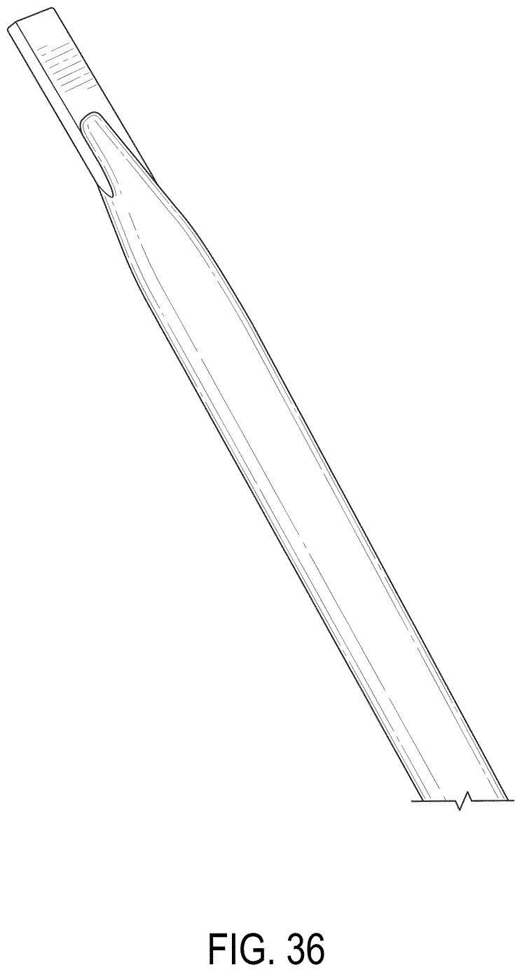

FIG. 35 depicts illustrative implementations of a cross-section of a dilator comparing different example taper angles.

FIG. 36 depicts a dilator disposed (e.g., overmolded) on an axial translation member.

Measurements depicted in the appendices are illustrative, and depict a specific contemplated embodiment, by way of example and not limitation. Additional dimensions and/or ratios therebetween are contemplated.

Although various embodiments are shown and described, other embodiments are contemplated. For example, some embodiments may be configured for use in percutaneous coronary intervention (PCI) procedures. For example, an LFD in a GEC may be configured to open blocked vasculatures (e.g., coronary arteries). Opening blocked vasculature or other restricted flow, may, for example, advantageously increase blood flow. Some embodiments may, for example, be configured to facilitate the bypass of calcification within vasculature (e.g., arteries). An LFD-equipped GEC may, for example, advantageously navigate through heavily calcified lesions, which might otherwise be challenging to treat.

Some embodiments may, for example, be configured for thrombus aspiration procedures. A LFD in a guide extension catheter may, for example, be configured to remove blood clots such as, by way of example and not limitation, in cases of ST-segment elevation myocardial infarction (STEMI). As another example, chronic total occlusion (CTO) PCI may be treated using an LFD in a GEC configured to assist in accessing and/or treating completely blocked arteries. Such embodiments may, for example, advantageously enable revascularization of previously untreatable occlusions.

Some embodiments may be configured, for example, suitable for use in transcatheter aortic valve replacement (TAVR).

Some embodiments may, for example, be configured to enable selective contrast injection. Selective contrast injection may, for example, reduce contrast use in patients (e.g., with renal compromise, with heart failure). An illustrative advantage includes, for example, reduced risk of contrast-induced nephropathy.

Some embodiments may, for example, advantageously enable successful engagement of aberrant-origin coronaries. For example, an LFD may be configured to facilitate procedures providing improved access to vasculature with unusual anatomical origins and/or geometric abnormalities.

In some examples, an LFD-equipped GEC may be configured for medical device (e.g., stent, graft) engagement and/or deployment. Some embodiments may, for example, be configured to engage and/or treat bypass grafts. For example, a smooth transition from the LFD to GEC may advantageously provide enhanced ability to access and/or intervene in grafted anatomy (e.g., vessels, nervous system, urinary system). An LFD may, for example, be employed during stent delivery, such as, for example, to place stents in narrowed and/or blocked arteries. An illustrative advantage may, for example, include secure and/or precise placement of stents to maintain vessel patency. An LFD may, for example, be configured for use in plaque modification (e.g., to modify plaque before stent placement).

In some embodiments, an LFD and/or GEC may, for example, be configured to advantageously deploy in removing plaque from arteries. For example, the LFD may enable enhanced maneuvering of the distal tip of the GEC to a treatment target.

Some embodiments may, for example, be configured for intravascular lithotripsy (IVL) and/or subsequent procedures. For example, an LFD-equipped catheter may advantageously be operated to enable non-invasive modification of calcified lesions such as, for example, to facilitate stent deployment.

In some examples, an LFD may, for example, be configured for use in balloon angioplasty. For example, a LFD-equipped GEC may be advantageously utilized to engage and widen narrowed and/or obstructed blood vessels.

Some embodiments may, for example, be configured to assist in advancing microcatheters for various procedures. For example, an LFD-equipped catheter may advantageously enhance maneuverability and/or stability of the microcatheter.

One or more embodiments may, by way of example and not limitation, be configured for use in thrombectomy procedures. An LFD may, for example, advantageously enable enhanced precision in bypassing and/or targeting a thrombus. Such embodiments may, by way of example and not limitation, advantageously enable restoration of blood flow and/or reduction or prevention of distal embolization.

In some embodiments, an LFD may be provided with a coating (e.g., hydrophilic). The coating may, for example, be applied to the outer surface of the dilator (e.g., dilator body 125, dilator tip 130). The coating may, for example, advantageously reduce friction during advancement through tortuous vessels (e.g., vasculature 100). The reduced friction may, for example, enable a tighter fitment of the LFD within the GEC and/or increased or preserved sensitivity and/or control by the operator.

In some implementations, a dilator (e.g., dilator 120) may be provided, for example, with multiple graduated diameter segments. The graduated segments may, for example, transition from a smaller diameter at the distal tip 130 to progressively larger diameters along at least a portion of the dilator body 125. This configuration may, for example, advantageously create progressive dilation. The progressive dilation may, for example, advantageously be used for tactile ‘depth measurement’ (e.g., the number of ‘pops’or ‘bumps’felt), such as when penetrating a restriction.

Some embodiments may, for example, include a dilator incorporating communication channels. Communication channels may, for example, include electrical channels (e.g. wires, embedded traces). The communication channels may, for example, include optical fibers. Communication channels may, for example, extend through the inner lumen 615 of the dilator (e.g., SAODLFD 605, such as disclosed with reference to FIGS. 6A-6E), through the dilator wall, and/or through a bladder (e.g., bladder 610). The communication channels may, by way of example and not limitation, advantageously enable real-time communication (e.g., visualization, sensing) with the vessel pathway during advancement, which may, by way of example and not limitation, improve navigation accuracy and/or safety.

In some implementations, a dilator (e.g., dilator 120) may be provided with one or more stiffening members. As an illustrative example, a nitinol core structure may be provided. The stiffening member(s) may, for example, be embedded within the dilator body 125. The stiffening member(s) may, for example, advantageously provide enhanced flexibility (e.g., without collapsing) and/or kink resistance during navigation through tortuous anatomy (e.g., vasculature 100).

Some embodiments may include a dilator (e.g., dilator 120) and/or GEC with a textured pattern (e.g., spiral-cut pattern), such as, for example, along its length. As an illustrative example, a spiral-cut pattern may be formed in the dilator body 125. The spiral-cut pattern may, for example, advantageously increase trackability through curved vessel segments while maintaining structural integrity. In some embodiments, a pattern may, for example, advantageously enable engagement with anatomy and/or obstructions (e.g., introducing rotation during advancement and/or advancement by rotation).

In some embodiments, a dilator may include a helical relief pattern with varying pitch. The varying pitch may be configured to create different expansion capabilities. This configuration may advantageously enable zone-specific expansion control along the length.

In some embodiments, a dilator may employ a cross-sectional geometry with a wave-shaped relief pattern. The wave pattern may be configured to create multiple contact points along the circumference. This configuration may advantageously provide decreased friction of the dilator during translation through the GEC.

Some implementations may include a dilator implementing nested relief patterns. The nested patterns may be configured to create progressive expansion zones. This arrangement may advantageously provide controlled deformation characteristics.

Some embodiments may include a dilator (e.g., dilator 120) with multiple internal lumens. The multiple lumens (e.g., replacing or additional to inner lumen 615) may, by way of example and not limitation, extend through the dilator body 125. A first lumen may, for example, accommodate a guide wire (e.g., guide wire 210). Additional lumens may, by way of example and not limitation, advantageously enable contrast injection and/or advancement of other devices (e.g., simultaneously).

In some implementations, a dilator (e.g., dilator 120) may, for example, be provided with variable stiffness zones. The variable stiffness zones may, for example, be formed by varying material composition and/or processing (e.g., curing) along the dilator body 125. In some examples, a proximal portion may be stiffer (e.g., to provide increased ‘push’), while the distal portion may be more flexible (e.g., decreasing stiffness from the tip 130 progressing along the tapered end 135). Such embodiments may, for example, advantageously increase navigability of tight, complex curves, while preserving sufficient stiffness for advancement at extended distances from a body entry point and/or penetration of obstructions.

Some embodiments may, by way of example and not limitation, include a dilator with an expandable tip and/or module (e.g., as disclosed at least with reference to FIGS. 5-7A). In some embodiments, the expandable tip may, for example, be configured at the distal tip 130 of the dilator (e.g., dilator 120). The expandable tip may, for example, advantageously create additional space for stent delivery in heavily calcified lesions (e.g., blockage 105) while maintaining a low initial crossing profile. Expandable tips and/or modules may, for example, advantageously enable selective control of fitment of the LFD within the GEC (e.g., especially at the distal end of the GEC).

Some embodiments may include a dilator (e.g., dilator 120) incorporating sensors, for example. Sensors may, for example, include pressure sensors. Sensors may, for example, include force sensors. Sensors may, for example, include flex sensors. Sensors may, for example, include proximity sensors. Sensors may, for example, motion and/or location sensors. The sensors may, for example, be disposed along the dilator body 125. The sensors may, for example, advantageously provide real-time feedback (e.g., about vessel wall resistance, about location, about dilator geometry) during advancement (e.g., through tortuous vasculature 100). In some embodiments, a fluid-pressure monitoring system may, for example, be provided (e.g., within dilator body 125). The monitoring system may, for example, advantageously provide feedback during navigation through vasculature 100.

In some implementations, a dilator (e.g., dilator 120) with a drug-eluting coating may be provided. The drug-eluting coating may be applied to the outer surface of the dilator body 125. As an illustrative example, drug-eluting coating may, for example, advantageously help prevent vessel spasm during procedures through local drug delivery.

Some embodiments may include a dilator (e.g., dilator 120) with integrated fluid distribution channels. The distribution channels may, for example, include fluid channels within the dilator body 125 (e.g., similar to inner lumen 615). The distribution channels may advantageously be configured to distribute fluid (e.g., liquid, gas) peripherally around a circumference of the dilator. Fluids may, for example, include drug delivery (e.g., vessel dilator). Fluids may, for example, include lubrication (e.g., advantageously reduce friction during advancement through tight lesions).

In some embodiments, a GEC (e.g., DFGEC such as disclosed at least with reference to FIG. 4) may, for example, include a stepped contour with multiple cross-sectional area (e.g., diameter) transitions progressively closer to the distal end. A stepped contour may, by way of example and not limitation, be configured such as disclosed at least with reference to contoured distal end 405. The stepped contour may, for example, advantageously provide enhanced stability during advancement through tortuous vasculature 100.

Some implementations of a GEC (e.g., DFGEC) may, for example, include a patterned texture (e.g., spiral-grooved contour), such as on the inner surface (e.g., of the distal end such as, for example, contoured distal end 405). The patterned contour may, for example, be configured to interact with a corresponding LFD (e.g., smooth, patterned outer surface). The pattern may, for example, interact with the LFD to center the LFD within the GEC. The pattern may, for example, be configured to reduce a surface area of contact between the LFD and the GEC (e.g., reduce friction, which may advantageously increase ability to induce movement of the LFD relative to the GEC). A spiral-grooved configuration may, for example, advantageously create controlled friction during relative movement between the dilator 120 and GEC 115. Some implementations may, for example, include a series of circumferential ridges on the inner surface of the GEC (e.g., the distal end such as near contoured distal end 405). A ridged configuration may, for example, advantageously enable precise tactile feedback during advancement procedures through vasculature 100. Some implementations may, for example, include a textured inner surface with microscopic patterns (e.g., on the inner surface of GEC 115). The textured surface may advantageously enhance controlled sliding between the dilator 120 and GEC 115, for example.

In some embodiments, an elastic (e.g., polymer) sleeve may be disposed at the distal end (e.g., near contoured distal end 405) of a GEC, for example. This configuration may, for example, advantageously provide adaptive diameter adjustment to accommodate different dilator sizes while maintaining the fitted transition 220. In some embodiments, a GEC may, for example, be provided with a bellows-like contour at the distal end (e.g., near contoured distal end 405) of a GEC. This configuration may, for example, advantageously provide for additional flexibility in multiple directions while maintaining a fitted transition (e.g., fitted transition 220) from the GEC distal end to the LFD.

Some implementations may include, for example, a series of longitudinal grooves in the distal end (e.g., near contoured distal end 405 of a GEC, on an outer surface of an LFD). The longitudinal grooves may, for example, advantageously enable fluid evacuation and/or friction control.

Some implementations may, for example, include a series of flexible fins at the interface between the dilator 120 and GEC 115. The flexible fins may, for example, advantageously create multiple contact points for enhanced stability.

In some embodiments, a coating (e.g., friction-reducing coating, hydrophilic coating) may be applied to the GEC (e.g., inner surface, outer surface). For example, a contoured surface (e.g., contoured distal end 405) may be coated. The coating may, for example, advantageously reduce friction during navigation through vasculature 100. The coating, when applied to the inner surface may, for example, advantageously enable more precise and/or more easily operated movement of the LFD relative to the GEC. Some implementations may include a nano-textured surface coating (e.g., on outer surface of dilator 120 or inner surface of GEC 115). The nano-textured coating may, for example, advantageously reduce surface friction during use. In some embodiments, a bioactive surface treatment may be applied (e.g., to dilator body 125). The bioactive treatment may, for example, advantageously promote lubricity during procedures through vasculature 100.

In some embodiments, a magnetic (e.g., electromagnetic) coupling system may, for example, be provided at an interface (e.g., at fitted transition 220). This system may advantageously provide, for example, controlled resistance during relative movement between the dilator 120 and GEC 115.

Some implementations may, for example, include a series of magnetic elements (e.g., disposed along dilator body 125 and GEC 115). The magnetic elements may advantageously provide adjustable resistance during relative movement between the dilator 120 and GEC 115.

Some implementations may include a dilator (e.g., dilator 120) with a piezoelectric element. The piezoelectric element may be incorporated into the dilator body 125. The piezoelectric element may advantageously enable active movement control during use through tortuous vasculature 100.

Some implementations may include a shape memory alloy interface (e.g., near fitted transition 220). The shape memory alloy may, for example, advantageously enable selective inducement (e.g., by the operator) of the LFD and/or GEC to adopt different (e.g., predetermined) geometries.

Some implementations may include a shape memory polymer (e.g., temperature-responsive polymer), such as in dilator body 125, tip 130. The temperature-responsive polymer may advantageously adjust flexibility during use based on environmental conditions within vasculature 100.

In some embodiments, a chamber (e.g., similar to bladder 610) may be disposed between components, for example. The chamber may, for example, be fluid-filled. The chamber may, for example, be gel-filled. The chamber may, for example, be foam filled. The chamber may, for example, be filled with particulates (e.g., microbeads). The chamber may, for example, advantageously act as a dynamic cushion during navigation through vasculature 100 and/or into/out of the GEC.

In some embodiments, a multi-durometer polymer transition may be provided (e.g., along dilator body 125). This configuration may advantageously provide graduated flexibility along at least a portion of the length of the device.

Some implementations may, for example, include reinforced (e.g., carbon-fiber, nanotube) section (e.g., in dilator body 125). The reinforcement may, for example, advantageously enhance structural integrity while maintaining flexibility. In some embodiments, the LFD and/or GEC may include a silicone-based composite material (e.g., in dilator body 125, in GEC 115). The silicone-based composite may, for example, advantageously enable enhanced elastic deformation while maintaining strength and/or biocompatibility. In some embodiments, a radiation-crosslinked polymer may be used (e.g., in dilator body 125). The radiation-crosslinked polymer may advantageously enhance durability while maintaining desired mechanical properties. Some implementations may include a ceramic-polymer hybrid material (e.g., in dilator body 125). The ceramic-polymer hybrid may advantageously provide wear resistance while maintaining flexibility.

In some embodiments, a braided reinforcement layer may be provided (e.g., within dilator 120, within body of GEC 115). The braided reinforcement may advantageously maintain structural integrity while allowing flexibility during navigation through tortuous anatomy.

Some implementations may, by way of example and not limitation, include a series of interlocking rings. In some implementations, at least some of interlocking rings may, for example, be located at an interface (e.g., between dilator 120 and GEC 115). The interlocking rings may advantageously enable, for example, segmented flexibility while maintaining engagement. Some implementations, for example, may include a series of interconnected flexible segments (e.g., along dilator body 125). The interconnected segments may advantageously provide graduated transition while maintaining strength during advancement through vasculature 100.

Some implementations may include, for example, alternating rigid and flexible segments (e.g., along dilator body 125). The alternating segments may advantageously create controlled bending while maintaining support during advancement. In some embodiments, a mechanical expansion system may employ interlocking (e.g., rigid, such as metal) segments (e.g., near fitted transition 220). The segments may be configured to slide outward radially when activated by a central control mechanism (e.g., axial translation member 140). This rigid expansion system may advantageously provide consistent and predictable engagement force.

In some embodiments, a helical support structure (e.g., within dilator body 125) may be provided, for example. The helical support structure may, for example, advantageously enable torsional stability during procedures through tortuous vasculature 100.

In some example embodiments, a composite mesh layer (e.g., within dilator body 125) may be provided. The composite mesh may, for example, advantageously provide reinforced flexibility during use through tortuous anatomy.

Some implementations may include, by way of example and not limitation, a shape memory alloy mesh structure (e.g., within dilator body 125). The mesh may, for example, be configured to automatically expand in response to a predetermined condition (e.g., body temperature) within vasculature 100. This configuration may, for example, advantageously provide automatic and/or selectively-controlled activation.

In some embodiments, a temperature control module (e.g., heater wire) may be provided. The temperature control module may, for example, advantageously enable an operator to selectively control response of the predetermined condition.

In some embodiments, a smart material transition zone may be provided (e.g., near fitted transition 220). The smart material (e.g., pressure-responsive, shear-responsive, elongation responsive, compression-responsive, temperature-responsive) may advantageously adapt (e.g., expanding, elongating, contracting, stiffening, relaxing) to anatomical forces during procedures through vasculature 100.

Some implementations may include a selective lumen fitment module (e.g., such as disclosed at least with reference to module 515) with a series of individual inflatable chambers (e.g., arranged circumferentially around the device). The multiple chamber design may, for example, advantageously enable precise control over the expansion pattern and contact pressure distribution.

In some embodiments, an expansion material may be provided in a fitment module (e.g., such as module 515, bladder 610, lumen fitment module 725). The expansion material may, for example, include hydrogel. The expansion material may, for example, be configured to gradually swell upon contact with moisture within vasculature 100. This arrangement may, for example, advantageously create a soft, conforming seal against the GEC wall and/or distal lumen aperture.

Some implementations may include, for example, a spiral-wound elastic element (e.g., within dilator body 125). The spiral configuration may advantageously, for example, enable compact storage while providing uniform circumferential expansion.

In some embodiments, a series of elastomeric fins may, for example, extend radially from the device body (e.g., dilator body 125). The flexible fins may be configured to naturally conform to irregular internal surfaces. Such embodiments may, for example, advantageously maintain consistent contact pressure.

Some implementations may, for example, include a magnetic expansion system (e.g., at or near fitted transition 220) incorporating multiple magnetic elements. The magnetic elements may be configured to repel each other when activated. This arrangement may, for example, advantageously provide precise electronic control over the expansion process.

In some embodiments, a braided mesh sleeve (e.g., such as disclosed at least with reference to FIG. 7A) may be configured, for example, as an expansion mechanism. The braided structure may be configured to contract longitudinally while expanding radially. This configuration may advantageously create selectively controlled engagement with the GEC wall.

Some implementations may include, by way of example and not limitation, a series of spring-loaded petals (e.g., near fitted transition 220). The mechanical petal system may advantageously provide reliable expansion with minimal complexity.

In some embodiments, a dual-layer balloon system (e.g., such as bladder 610) may be configured as an expansion mechanism. The inner balloon may, for example, provide primary expansion. The outer balloon may include a textured surface. This configuration may advantageously provide enhanced grip and/or selective control.

In some embodiments, a bio-compatible foam material may be provided as an expansion medium. The foam may, for example, be configured to expand gradually when exposed to specific conditions. This arrangement may advantageously create a gentle fit (e.g., not exceeding a predetermined engagement force controlled by foam composition). The foam may, for example, be compressible (e.g., by applying a withdrawal force to the dilator sufficient to compress the foam. In some embodiments the foam may, for example, be shape memory foam. The foam may, for example, be selectively collapsible (e.g., by changing environmental variables such as temperature, electrical properties).

Some implementations may include an electroactive polymer system forming the expansion mechanism. The polymer may, for example, be configured to change shape in response to electrical signals. This configuration may advantageously enable precise electronic control of the fitting process.

In some embodiments, a bladder implementation may, for example, include a series of interconnected fluid chambers arranged in a helical pattern around the inner lumen. The helical arrangement may advantageously enable enhanced flexibility during bending movements while maintaining a fitted transition.

Some implementations may include a bladder design incorporating multiple isolated compartments separated by elastic membranes. The compartmentalized structure may advantageously allow for localized diameter adjustments along different sections (e.g., of a SAODLFD).

In some embodiments, a bladder configuration may employ a double-wall construction with an outer elastic layer and inner semi-rigid layer. This layered construction may advantageously provide controlled expansion characteristics while maintaining the inner lumen.

Some implementations may include support elements implemented as a mesh network of interwoven flexible polymer strands. The mesh structure may advantageously enable multi-directional support distribution within the bladder.

In some embodiments, support elements may be created as a series of curved arch segments spaced along the length. The arch design may advantageously provide radial support strength while maintaining flexibility. Some implementations may include support elements formed as spiral coils embedded within the bladder walls. The spiral configuration may, for example, advantageously enable both longitudinal flexibility and radial support. In some embodiments, support elements may be constructed as honeycomb structures (e.g., expandable), for example. The honeycomb geometry may, for example, advantageously provide good strength-to-weight characteristics within the bladder.

Some implementations may include a bladder(s) filled with a magnetorheological fluid that changes viscosity in magnetic fields. The variable viscosity may advantageously enable field-controlled stiffness adjustment (e.g., of an LFD such as an SAODLFD).

In some embodiments, a fitment module may include, for example, elastomeric bands integrated with rigid expansion elements. The elastomeric bands may, for example, be configured to ensure gentle contact with surrounding surfaces. This implementation may advantageously provide natural return force for device retraction.

In some embodiments, a lumen fitment module may be implemented as a spherical bulb shape at the distal end. The spherical shape may be configured, for example, to provide a tighter fitment between the LFD and GEC. This configuration may advantageously provide uniform pressure distribution around the circumference of the seal.

Some implementations may include a lumen fitment module implemented as a tapered cone configuration. The tapered cone may, for example, be configured with a gradual taper angle from proximal to distal ends. This arrangement may advantageously help guide insertion while maintaining consistent wall thickness.

In some embodiments, a lumen fitment module may be manufactured as a single molded piece with the LFD body using multi-shot injection molding. The single-piece construction may be configured to provide seamless transition between components. This configuration may, for example, advantageously eliminate potential separation points.

Some implementations may include a lumen fitment module implemented with a series of circumferential ridges along its outer surface. The ridges may be configured to engage with the inner surface of the GEC. This arrangement may advantageously decrease surface area contact (e.g., which may increase navigability and/or operator control).

In some embodiments, a lumen fitment module may be manufactured from a softer durometer material than the main body (e.g., dilator body 125), for example. The softer material may be configured to deform under contact pressure. This configuration may, for example, advantageously enhance conformability to irregular surfaces.

Some implementations may include a lumen fitment module implemented with an internal reinforcement structure including radial supports. The radial supports may be configured to maintain structural integrity under compression. This arrangement may advantageously prevent collapse while maintaining flexibility.

In some embodiments, a lumen fitment module may be manufactured using an over-molding process (e.g., where the hub is molded directly onto the body). The over-molding process may, for example, be configured to create a chemical bond between components. This configuration may, for example, advantageously create an extremely strong connection.

Some implementations may include a lumen fitment module implemented with a textured external surface pattern. The surface texturing may be configured to increase friction coefficient. This arrangement may advantageously improve retention within the GEC.

Some implementations, for example, may include a dilator incorporating thick and thin wall sections (e.g., alternating). The varying wall thickness may be configured to create zones of different flexibility. Such arrangements may, for example, advantageously provide controlled deformation characteristics. In some embodiments, a lumen fitment module may be manufactured with varying wall thickness sections. The wall thickness variations may be configured along the module. Such configurations may, for example, advantageously optimize both flexibility and strength.

Some implementations may include a lumen fitment module, for example, implemented with an accordion-style expandable section. The expandable section may be configured to compress and expand axially. This arrangement may advantageously accommodate dimensional variations while maintaining seal. In some embodiments, a dilator may employ a star-shaped cross-sectional relief geometry. The star shape may be configured to create multiple expansion zones. This configuration may advantageously maintain structural integrity while enabling controlled deformation.

Some implementations may include a lumen fitment module implemented with an elliptical cross-section profile. The elliptical shape may be configured with different major and minor axes. This arrangement may advantageously provide better resistance to rotational forces.

In some embodiments, an LFD and/or GEC may be manufactured using ultrasonic welding to join components. The ultrasonic welding may, for example, be configured to create molecular bonds at the joint interfaces. This configuration may advantageously create a hermetic seal at seams and/or joints.

In some embodiments, a dilator may implement, for example, a collar with variable diameter segments along its length. The variable diameter segments may be configured to provide selective engagement points. This configuration may advantageously enable controlled engagement with the GEC at different positions.

Some implementations may, for example, include a dilator including a circumferential engagement translation module with textured gripping surfaces. The textured surfaces may be configured to increase friction coefficient. This arrangement may advantageously enhance control during axial advancement.

In some implementations, a dilator with one or more visualization markers may be provided. The marker(s) may, for example, be radiopaque. The marker(s) may, for example, be embedded in and/or attached to the dilator tip 130 (e.g., at or near the tapered end 135). The marker(s) may, for example, advantageously enhance visibility (e.g., under fluoroscopy), such as enabling precise positioning during procedures. In some embodiments, markers (e.g., radiopaque) may be spaced according to a predetermined pattern (e.g., equally spaced). The predetermined spacing pattern may, for example, advantageously enable measurement (e.g., of a lesion and/or treatment area length).

In some embodiments, a dilator may, for example, incorporate a dilator tail with strain measurement markings (e.g., integrated). The strain markings may be configured, for example, to provide visual indication of deformation. This configuration may advantageously enable monitoring of dilator performance during use.

In some embodiments, a dilator may employ a CETM with visual position indicators. The position indicators may be configured to provide advancement feedback. This configuration may advantageously enable monitoring of procedural progress.

Some implementations may include a dilator with multiple radially-distributed lumen access ports. The multiple ports may be configured to enable simultaneous fluid communication at different positions. This arrangement may advantageously provide multiple access points around the circumference.

Some implementations may, for example, include a dilator with a lumen access port incorporating a self-sealing elastomeric membrane. The elastomeric membrane may, for example, be configured to automatically seal when not in use. This arrangement may advantageously maintain lumen integrity during procedures.

Some implementations may include a dilator with a lumen access port with integrated flow control valve. The flow control valve may be configured to regulate fluid flow. Such examples may advantageously enable precise control of fluid introduction or extraction.

In some embodiments, a dilator may implement a CETM with programmable engagement force control (e.g., internal, external control module). The force control system may be configured to adjust advancement pressure. This configuration may advantageously dynamically adjust force application based on encountered resistance.

In some embodiments, a tapered (e.g., conical) friction fit module (e.g., as shown in Appendix B may be implemented with matching tapered surfaces between axial interdigitation modules. The tapered surfaces may be configured to self-align during engagement. This configuration may advantageously facilitate proper alignment between the LFD and CETM during coupling operations.

Some implementations may include, for example, a bayonet mount module implemented with slots (e.g., L-shaped) engaging corresponding pins between axial interdigitation modules. The bayonet mechanism may, for example, be configured to provide quarter-turn locking engagement. This arrangement may advantageously enable quick connect/disconnect capability while maintaining secure coupling during use.

In some embodiments, a dilator may include a bulbous shape at the distal end (e.g., at the location depicted for tapered tip 130). The bulbous shape may be configured to provide an expanded diameter region. This configuration may, for example, reduce drag of the dilator body.

Some examples may include a dilator and/or GEC with a passive adjustment (e.g., expansion) mechanism, such as disclosed at least with reference to FIGS. 4-6E. The passive adjustment mechanism may be configured to expand in response to mechanical forces. This arrangement may, for example, advantageously provide automatic diameter adjustment during advancement.

In some embodiments, a dilator may include, for example, an active expansion mechanism (e.g., as disclosed at least with reference to FIG. 7A). The active expansion mechanism may be configured to expand in response to operator control. This configuration may advantageously enable selective diameter adjustment during procedures.

Some implementations may include a dilator with a foldable expansion structure. The foldable structure may be configured to expand from a collapsed state. An expanding structure may, for example, advantageously provide a low profile during insertion while enabling selective expansion.

In some embodiments, for example, a dilator may include a laser-cut hypo tube transition to flat wire. The transition may be configured proximal of a proximal end of the dilator body 125. Such embodiments may advantageously provide enhanced flexibility while maintaining pushability.

In some embodiments, an axial translation member (e.g., push tube, push rod) may, for example, be constructed at least partially of tempered metal (e.g., stainless steel).

Some implementations may include a dilator with a stretchable distal tip. The stretchable tip may be configured to elastically deform. This arrangement may advantageously enable accommodation of different device sizes (e.g., passed through the inner lumen 615 of the LFD).

Some implementations may include a dilator with a multi-layer construction. For example, the construction may include polytetrafluoroethylene (PTFE). The construction may, for example, include polyamide. The construction may, for example, include coil (e.g., spring metal). The multi-layer construction may be configured to provide specific mechanical properties. Such embodiments may, for example, advantageously optimize flexibility, strength, and lubricity.

In some embodiments, a dilator and/or GEC may be manufactured at least partially from polypropylene (PP). PP may, for example, provide a high-stiffness polymer advantageously providing substantial tensile strength. PP may have a lower lubricity then other polymers disclosed herein, but superior lubricity compared to conventional elastomers. PP may, for example, advantageously provide excellent pigment and/or radiopaque filler acceptance properties. PP may, for example, advantageously be highly cost-effective.

Some embodiments may be at least partially manufactured of polyethylene (PE), such as high-density polyethylene (HDPE) and/or low-density polyethylene (LDPE). Both are moderately stiff polymers, which may provide respective durometers of approximately 50D and 40D. These materials, either individually or as a blended composition, may be employed in the fabrication of dilators and/or sheaths. Polyethylene polymers may, for example, advantageously provide lubricity. PE may, for example, be readily amenable to incorporation of color pigments and/or radiopaque fillers. PE may, for example, be a cost-effective material.

Some embodiments may, for example, be made partially or completely of fluorinated ethylene propylene (FEP). FEP may, for example, provide a very soft polymer. FEP may, for example, be advantageously incorporated in sheath components, such as where lubricity may be particularly important. FEP is a melt-extrudable fluorinated polymer, which may provide high lubricity. FEP may be more difficult to incorporate radiopaque fillers and/or color pigments. FEP may, for example, have a higher cost relative, for example, to polypropylene and polyethylene.

Ethylene tetrafluoroethylene (ETFE), characterized by higher stiffness than FEP, may be used, for example, in producing sheath components. ETFE may, for example, provide enhanced lubricity and stiffness. ETFE may, for example, exhibit high natural lubricity among extrudable polymers. ETFE may, for example, have similar cost and pigment and/or filler attributes to FEP.

In some embodiments, a dilator and/or catheter (e.g., GEC) may include a thin wall construction of less than 0.01 inches. In some embodiments, the wall thickness may, by way of example and not limitation, be less than or equal to 0.004 inches. In some embodiments, a thin-walled GEC may, for example, be structurally supported (e.g., to prevent axial collapse and/or kinking) during advancement by an LFD. The thin wall construction may be configured, for example, to increase inner lumen diameter. Such examples may, for example, advantageously enable passage of larger devices while maintaining a small outer profile.

In some example embodiments, a dilator may include a shapeable tip. The shapeable tip may, for example, be configured to be formed into different curves. This configuration may advantageously enable customization for different anatomies. The shapeable tip may, for example, be pre-shapeable (e.g., by a physician). In some embodiments, a dilator may include a shapeable tip section. The shapeable tip section may be configured to be manually formed into different curves. For example, the shapeable tip may include a malleable material. The malleable material may enable a physician to customize the curve according to specific patient anatomy. This arrangement may advantageously provide procedural flexibility while maintaining a fitted transition between the dilator and GEC.

A shapeable tip may, for example, be dynamically shapeable (e.g., by a steering control unit operated by the physician). The shape may, for example, be controlled by steering wires coupled to the tip. The shape may, for example, be controlled by environmental factors (e.g., a shape memory tip). Some implementations may, for example, include a dilator with a steerable tip section. The steerable tip may, for example, be configured to be actively deflected. Such example arrangements may, for example, advantageously provide enhanced navigation control during procedures. Some implementations, as an illustrative example, may include a dilator with a shape memory material in the preshaped section. The shape memory material may be configured to assume a predetermined curve at body temperature. For example, the shape memory material may include nitinol. The nitinol may be processed to transition to the desired curve when warmed to body temperature. This configuration may advantageously provide consistent curve formation during procedures.

Some implementations may include a dilator with a preshaped configuration. The preshaped configuration may, for example, be configured to match specific anatomical curves. Such example embodiments may, for example, advantageously enhance navigation through tortuous vasculature. Some implementations may include a dilator with multiple preshaped configurations. The multiple preshaped configurations may be configured for different anatomical regions. As an illustrative example, a first preshaped configuration may be configured for coronary vessels. A second preshaped configuration may, by way of example and not limitation, be configured for peripheral vessels. This configuration may advantageously enable selection of a desired shape for specific procedures and/or anatomical targets.

In some embodiments, a GEC (e.g., GEC 115) may, for example, be configured as a guide extension liner. A guide extension liner embodiment may be configured as a flexible tube configured to facilitate guide extension within a patient's vasculature. Some embodiments may, for example, have a thin-walled construction that enhances the inner lumen diameter while maintaining a compact outer profile. Various embodiments may be configured with any one or combination of the features related to GEC disclosed herein.

Some embodiments may encompass various components, display technologies, chip technologies, interface technologies, power supply technologies, power storage technologies, server architectures, personal device architectures, portable personal computing devices, and/or software architectures.

For example, processor(s) may include central processing units (CPUs). CPUs may, for example, serve as the ‘brain’ of computer systems, such as by executing instructions and/or processing data, for example. A processor may, for example, include an arithmetic logic unit (ALU), a control unit, and/or numerous registers. Processor(s) may, for example, include graphics processing units (GPUs). GPUs may, for example, be configured to render images, videos, and/or animations. GPUs may, for example, advantageously provide greater speed for parallel processing tasks. Accordingly, GPUs may, for example, be advantageously used for tasks requiring intensive graphical computations.

Some embodiments may, for example, include application-specific integrated circuits (ASICs). ASICs may, for example, be custom-designed circuits (e.g., chips) tailored for specific applications. ASICs may, for example, provide high performance and efficiency.

Some embodiments may, for example, include field-programmable gate arrays (FPGAs). FPGAs may be configured, for example, as reconfigurable chips that can be programmed to perform various functions. FPGAs may, for example, advantageously be used in prototyping and/or specialized computing tasks.

Microprocessors may, for example, be configured as general-purpose chips. Microprocessors may, for example, execute instructions from software applications. As such, microprocessors may advantageously be utilized, for example, in a wide range of devices, from desktop computers to embedded systems.

Memory modules, may, for example, include volatile memory (RAM) and/or non-volatile memory (ROM). RAM may, for example, be used for temporary data storage. ROM may, for example, store firmware and/or system-level software.

Storage devices may include, for example, hard disk drives (HDDs), solid-state drives (SSDs), and/or optical drives. Storage devices may, by way of example and not limitation, store a device operating system(s), applications, and/or user data.

Input/output (I/O) interfaces may include, by way of example and not limitation, data ports, graphics ports, and/or audio ports. Data ports may include, for example, USB ports (e.g., USB-A, USB-C, USB-Mini, USB-Micro), Ethernet (e.g., RJ45), SATA ports, serial and/or parallel ports. Graphics ports may include, for example, HDMI ports, VGA ports, and/or Display Port ports. Some ports may, for example, be multi-purpose (e.g., USB-C may carry audio, graphics, and/or other data). Audio ports may include, for example, audio jacks. I/O interfaces may, for example, facilitate communication between the computer and peripheral devices.

Display technologies may include, by way of example and not limitation, liquid crystal displays (LCDs). LCDs may, for example, be used in monitors, laptops, and/or televisions. LCDs may, for example, modulate light passing through liquid crystals to produce images. Display technologies may include, for example, light emitting diode (LED) displays. LED displays may, for example, utilize an array of LEDs for back lighting and/or as a primary display technology. LED displays may, for example, offer enhanced brightness and/or color accuracy (e.g., compared to LCDs). Organic light emitting diode (OLED) displays, for example, may employ organic compounds that emit light when an electric current is applied. OLED displays may, for example, advantageously provide high contrast ratios and/or fast response times. Display technologies may, for example, include electronic paper displays (EPDs), which may also be known as e-ink displays. EPDs may, for example, be employed in e-readers. EPDs may, for example, deliver a paper-like reading experience, reduce eye strain, and/or reduce power consumption.

Interface technologies may, for example, encompass various devices that enable user interaction with a computer system. Some embodiments may, for example, include a mouse(s). A mouse may, for example, be configured as a pointing device that detects motion and translates it into cursor movement on the screen. As such, a mouse may, for example, advantageously allow a user to interact with graphical user interfaces.

Keyboards may, for example, be configured for text entry and/or command execution. A keyboard may, for example, include multiple keys (e.g., arranged in a standard layout).

Touch inputs may, for example, enable direct interaction with a display or other input device through gestures such as tapping, swiping, and pinching.

Some embodiments may, for example, include an audio capture device(s), such as a microphone(s). for example, a device may be configured to record voice inputs. Some embodiments may, for example, leverage voice recognition technology, allowing users to control devices and/or enter text using spoken commands.

Additional interface technologies which may be included in some embodiments may, by way of example and not limitation, include track pads, joysticks, styluses, and/or game controllers.

Various embodiments may include one or more power supply and/or storage technologies. For example, power supplies may convert electrical power from an outlet into usable power for a device's components. A power supply may, for example, include one or more transformers, rectifiers, and/or regulators. Batteries may, for example, advantageously provide portable power for devices such as laptops, smartphones, and tablets. Batteries of one or more chemistries may be used, including, by way of example and not limitation, lithium-ion and/or nickel-metal hydride.

In some embodiments, devices disclosed herein may be configured as and/or connected in a server architecture. A server architecture may, for example, be configured to advantageously provide scalable computing resources, such as in enterprise environments, for example. Some embodiments may, for example, include blade servers. Blade servers may, for example, be configured as modular servers that fit into a chassis, which may advantageously allow for high-density computing and/or optimize space and/or power efficiency in data centers. Rack servers may, for example, be configured to be mounted in standardized racks. Rack servers may, for example, advantageously provide scalable computing resources. Cloud servers may, for example, include virtualized servers, which may be hosted in data centers. Cloud servers may, for example, advantageously offer flexible and/or scalable resources to users over the internet.

In some embodiments, devices disclosed herein may be configured as and/or connected to a personal device architecture, for example. Personal device architectures may include, for example, desktop computers. A desktop computer may, for example, include a tower, monitor, keyboard, and mouse, and may be used, for example, for a wide range of applications, from office work to gaming. Laptops may, for example, be configured as portable computers. The portable computers may, for example, integrate a display, keyboard, and position input (e.g., track pad) into a single unit. Laptops may, for example, be used for mobile computing and may, for example, perform many of the same tasks as desktops. Portable personal computing devices may, by way of example and not limitation, include smartphones. Smartphones may be configured, for example, as compact devices that combine computing capabilities with telecommunication functions. These devices may include, by way of example and not limitation, touchscreens, cameras, and/or various sensors. Portable personal computing devices may include, for example, smartwatches. Smartwatches may, for example, be configured as wearable devices. Smartwatches may, for example, provide notifications, fitness tracking, and/or other functionalities. Smartwatches may, for example, be configured to pair with smartphones and/or other computer(s) for extended capabilities. Portable personal computing devices may, for example, include tablets. Tablets may, for example, be configured as portable devices with touchscreens larger than smartphones. Tablets may, for example, advantageously be used for tasks such as web browsing, media consumption, and/or productivity applications.

Engines and/or modules disclosed herein may be configured in one or more software architectures. Software architectures may, for example, include operating systems. Operating systems may, for example, manage hardware resources and/or provide a platform for running applications.

Software architectures may, for example, include application software. Application software may include, for example, programs designed for specific tasks.

Software architectures may, for example, include middleware. Middleware may, for example, be configured to provide services to software applications beyond those offered by the operating system. Middleware may, for example, include components such as web servers, database management systems, and/or message brokers.

Software architectures may include, for example, firmware. Firmware may, for example, be configured as low-level software embedded in hardware devices. Firmware may, for example, controls functions of the hardware devices. Firmware may, by way of example and not limitation, be stored in ROM and/or flash memory.

Software architectures may, for example, include virtualization technology. Virtualization technology may, for example, be configured to allow multiple virtual machines to run on a single physical machine. Virtualization technology may, for example, advantageously enable efficient resource utilization and/or isolation.

Various embodiments may, for example, include connection and/or communication technologies. Such technologies may, by way of example and not limitation, be configured to facilitate the exchange of data across various distances and/or environments. Long-range communication technologies may, by way of example and not limitation, include cellular networks, satellite communications, and/or broadband internet connections. Cellular networks, such as 4G LTE and 5G, may advantageously provide wireless connectivity over large areas. Cellular networks may, for example, enable devices (e.g., mobile devices) to access the internet, make calls, and/or otherwise transmit data. Satellite communications may, for example, advantageously provide global coverage, which may be particularly useful in remote and/or underserved regions where terrestrial infrastructure is limited. Broadband internet connections may include, by way of example and not limitation, fiber-optic, DSL, and/or cable. Broadband may, for example, advantageously provide high-speed internet access to devices such as for activities including streaming, online gaming, and/or remote work.

Local communication technologies may, for example, encompass methods for connecting devices within a limited area, such as a home, office, and/or campus. Local communication technologies include, for example, Wi-Fi. Wi-Fi may, for example, connect device(s) to a wireless local area network (WLAN) and/or access the internet and/or share resources (e.g., printers, storage). Wired communication technology, such as Ethernet, may, for example, advantageously provide reliable and/or high-speed connections between devices in a local network, such as, by way of example and not limitation, desktops, servers, and/or network switches. Power-line communication (PLC) may, for example, enable data transmission over existing electrical wiring. PLC may, for example, advantageously provide an alternative for connecting devices in locations where Wi-Fi signals may be weak or unreliable.

Near field communication (NFC) and BLUETOOTH are examples of short-range communication technologies. Short-range communication technologies may, by way of example and not limitation, be configured to connect devices within a few centimeters to several meters. NFC may, for example, include wireless technology configured to enable contactless communication between devices. NFC may, for example, be configured for use with mobile payments, access control, and/or data transfer. Its short range may, for example, advantageously enhance security by requiring close proximity for communication. Bluetooth may, for example, provide wireless connectivity over a range of meters (e.g., up to 100 meters). Bluetooth may, for example, advantageously be deployed in implementations connecting peripherals such as keyboards, mice, headphones, and/or wearable devices, and/or for transferring files between devices.

Various embodiments may, for example, include a dilator with longitudinally-extending relief.

In some embodiments, a dilator may be provided that includes a dilator body extending along a longitudinal axis from a proximal end to a distal end, with a tapered tip disposed at the distal end. The dilator body may include a longitudinally-extending relief that runs along at least a portion of the length of the dilator body. The longitudinally-extending relief may be configured such that a transverse cross-section of the dilator body, taken perpendicular to the longitudinal axis, is less than a full circle. This relief may, for example, be configured to accommodate an axial translation member disposed along an outer surface of the dilator body, thereby allowing the axial translation member to be received within the profile of the dilator body rather than protruding beyond it. This configuration may, for example, reduce the overall outer profile of the dilator-and-translation-member assembly, facilitating navigation through narrow lumens such as those of a guide extension catheter.

In some embodiments, the transverse cross-section of the dilator body may define a D-shaped cross-sectional geometry. In such embodiments, the longitudinally-extending relief may include a flat relief along one side of the dilator body. The flat relief may, for example, provide a planar surface against which the axial translation member may be seated, enabling a low-profile interface between the dilator body and the axial translation member. This D-shaped geometry may, for example, simplify manufacturing while providing a well-defined seating surface for the axial translation member.

In some embodiments, the transverse cross-section of the dilator body may define a cross-sectional geometry that includes a curved relief. Rather than a flat surface, the relief may follow a curved contour that may, for example, be shaped to conform to the outer surface of a cylindrical axial translation member such as a push rod or hypotube. This curved relief may, for example, provide increased contact area between the dilator body and the axial translation member, potentially improving load distribution and reducing stress concentrations during advancement.

In some embodiments, the transverse cross-section of the dilator body may, for example, define a cross-sectional geometry including a polygonal relief. The polygonal relief may, for example, include one or more angled facets that together define a recessed region along the outer surface of the dilator body. Such a polygonal geometry may, for example, provide a defined channel for the axial translation member while also offering structural rigidity along the relieved portion of the dilator body.

In some embodiments, the transverse cross-section of the dilator body may define a cross-sectional geometry including a notched relief. The notched relief may, for example, be formed as a discrete channel or groove extending longitudinally along the outer surface of the dilator body. The notched relief may, for example, be sized and shaped to receive the axial translation member in a closely fitting manner, thereby constraining lateral movement of the axial translation member relative to the dilator body during advancement through a guide extension catheter.

In some embodiments, the dilator may further include a collar disposed on the dilator body. The collar may have an outer diameter configured to provide a fitted engagement within an inner lumen of a guide extension catheter. The collar may, for example, be formed from a compliant or semi-rigid material that engages the inner wall of the guide extension catheter to reduce or eliminate fishmouthing at the distal mouth of the guide extension catheter. This fitted engagement may, for example, improve pushability and trackability of the dilator-catheter assembly through tortuous anatomy.

In some embodiments, the collar may be disposed proximally to the tapered tip of the dilator body and may be configured to substantially occlude the inner lumen of the guide extension catheter at the distal mouth of the guide extension catheter. By substantially occluding the distal mouth, the collar may, for example, help prevent the distal lip of the guide extension catheter from splaying outward—a condition commonly referred to as fishmouthing—when the dilator is advanced through a vascular lesion or other obstruction. This configuration may, for example, enable the guide extension catheter to track closely behind the dilator during advancement through calcified or otherwise resistant lesions.

In some embodiments, the axial translation member may be disposed within the longitudinally-extending relief and may extend proximally from the proximal end of the dilator body. By seating the axial translation member within the relief, the combined outer profile of the dilator body and axial translation member may, for example, be reduced relative to configurations in which the axial translation member is laminated on the outer surface of the dilator body without a corresponding relief. This arrangement may, for example, allow a larger-diameter dilator body to be used within a given guide extension catheter lumen, improving the fitted transition between the dilator and the catheter.

In some embodiments, the axial translation member may include a push rod, and the longitudinally-extending relief may be configured to accommodate a weldment that protrudes radially outward from the push rod. Weldments, such as those formed at a junction between the push rod and a hypotube or other coupling element, may create localized protrusions along the axial translation member. By sizing and shaping the longitudinally-extending relief to receive such weldments, the dilator body may, for example, accommodate these protrusions without increasing the overall outer diameter of the assembly. This configuration may, for example, help prevent the weldment from creating a step or bump that could snag on the inner wall of the guide extension catheter during advancement.

In some embodiments, the dilator may be used in combination with a guide extension catheter having a lumen extending from a proximal end to a distal mouth. The dilator body may be receivable within the lumen of the guide extension catheter such that both the longitudinally-extending relief and the axial translation member are disposed within the lumen of the guide extension catheter. Because the axial translation member may be received within the longitudinally-extending relief rather than being laminated on the outer surface of the dilator body outside of the relief, the outer diameter of the dilator body may, for example, be increased relative to a configuration in which the axial translation member is laminated externally. This increased outer diameter may, for example, provide a tighter, more fitted interface between the dilator body and the inner lumen of the guide extension catheter, reducing the gap at the distal mouth and thereby reducing or eliminating fishmouthing.

In some embodiments, the dilator body may be configured to form a fitted transition between the outer surface of the dilator body and the distal mouth of the lumen of the guide extension catheter. The fitted transition may, for example, be achieved by selecting a dilator body outer diameter that closely approximates the inner diameter of the guide extension catheter lumen at the distal mouth. This snug fit may, for example, help maintain structural continuity between the dilator and the guide extension catheter during advancement through vascular obstructions, reducing the likelihood of the guide extension catheter snagging or failing to track the dilator.

In some embodiments, the transverse cross-section of the dilator body may define a wave-shaped relief pattern. The wave-shaped relief pattern may be configured to provide contact points distributed along the circumference of the dilator body. These multiple contact points may, for example, engage the inner wall of the guide extension catheter at discrete locations around the circumference, potentially reducing the total contact area and thereby decreasing friction during translation of the dilator through the guide extension catheter. This configuration may, for example, facilitate smooth advancement of the dilator while maintaining sufficient engagement with the guide extension catheter to support pushability and positional stability.

Some embodiments may, for example, include a dilator with a unitary bladder.

In some embodiments, a dilator may include a dilator body extending along a longitudinal axis from a proximal end to a distal end. The dilator body may have an outer wall and an inner wall that together define a guidewire lumen extending through the dilator body along its length. A tapered tip may be disposed at the distal end of the dilator body to facilitate atraumatic advancement through anatomical structures. A unitary bladder may define a closed void disposed between the outer wall and the inner wall of the dilator body. The closed void may be fluidly isolated from the guidewire lumen, such that the guidewire lumen remains unobstructed and available for guidewire passage. The unitary bladder may extend radially from the dilator body at a distal region of the dilator body proximally of the tapered tip. The unitary bladder may be configured to deform so as to adjust an outer diameter of the dilator body at the distal region, thereby enabling the dilator to accommodate varying anatomical or device constraints during use.

In some embodiments, support elements may be disposed within the closed void of the unitary bladder. The support elements may, for example, be configured to provide structural support to the bladder while still permitting controlled deformation of the unitary bladder. This arrangement may, for example, help prevent undesired collapse or over-expansion of the bladder during advancement through a guide extension catheter or anatomical passage, while preserving the bladder's ability to adjust the outer diameter of the dilator body as needed.

In some embodiments, the support elements within the closed void may, for example, be configured as longitudinally-extending ribs. The ribs may, for example, be oriented along the length of the unitary bladder and distributed around the interior of the closed void. This rib configuration may, for example, provide directional stiffness along the longitudinal axis while permitting radial deformation of the bladder, thereby enabling controlled diameter adjustment without compromising the structural integrity of the dilator body.

In some embodiments, the support elements within the closed void may, for example, include a mesh network of interwoven flexible polymer strands. The mesh network may, for example, be distributed throughout the interior of the closed void. This configuration may, for example, provide more uniform structural support across the bladder volume, distributing deformation forces more evenly and reducing the likelihood of localized stress concentrations during radial expansion or compression of the unitary bladder.

In some embodiments, the support elements within the closed void may include curved arch segments spaced along a length of the unitary bladder. The arch segments may, for example, be arranged at intervals within the closed void to provide periodic structural reinforcement. This configuration may, for example, create a series of controlled deformation zones between adjacent arch segments, enabling graduated and predictable radial expansion characteristics along the length of the distal region of the dilator body.

In some embodiments, the unitary bladder may circumscribe the inner wall of the dilator body such that the closed void extends around an entirety of the circumference of the dilator body at the distal region. This circumferential arrangement may, for example, provide more uniform radial expansion in multiple directions, enabling the dilator body to form a symmetric, well-fitted interface with the inner wall of a guide extension catheter or other surrounding structure regardless of rotational orientation.

In some embodiments, the closed void of the unitary bladder may be at least partially filled with a material. Suitable fill materials may, for example, include a fluid, a solid material, a gel, a foam, particulates, and/or a magnetorheological fluid. The selection of fill material may, for example, be tailored to achieve desired deformation characteristics, stiffness profiles, and/or responsiveness to external stimuli. For example, a magnetorheological fluid fill may, for example, enable active, externally-controlled stiffness adjustment of the bladder in response to an applied magnetic field.

In some embodiments, the closed void of the unitary bladder may, for example, be hollow, containing no fill material. A hollow closed void may, for example, reduce the overall weight and profile of the dilator while still enabling the bladder to deform and adjust the outer diameter of the dilator body through elastic deformation of the bladder walls themselves.

In some embodiments, the dilator may be used in conjunction with a guide extension catheter having a lumen extending from a proximal end to a distal mouth. The dilator body may be receivable within the lumen of the guide extension catheter. In this configuration, the unitary bladder may, for example, be configured to self-adjust the outer diameter of the dilator body so as to form a fitted transition between the outer wall of the dilator body and the distal mouth of the lumen of the guide extension catheter. This self-adjusting fitted transition may, for example, reduce or eliminate fishmouthing at the distal mouth of the guide extension catheter, thereby facilitating smoother passage of the dilator and reducing the likelihood of snagging during vascular procedures.

In some embodiments, the unitary bladder may, for example, be configured to interface with guide extension catheters having different lumen diameters. The bladder may, for example, self-adjust the outer diameter of the dilator body to conform to each respective lumen diameter encountered. This adaptive capability may, for example, allow a single dilator to be used across a range of guide extension catheter sizes, reducing the need for multiple dedicated dilator configurations and simplifying procedural preparation.

In some embodiments, the unitary bladder may include a double-wall construction including an outer elastic layer and an inner semi-rigid layer. The outer elastic layer may, for example, accommodate radial deformation and conformance to surrounding structures, while the inner semi-rigid layer may, for example, provide controlled expansion characteristics and help maintain the integrity of the guidewire lumen. This layered construction may, for example, balance the competing demands of flexibility and structural support within the bladder.

In some embodiments, the unitary bladder may include isolated compartments separated by elastic membranes. The isolated compartments may, for example, be configured to permit localized diameter adjustments along different sections of the distal region of the dilator body. This compartmentalized structure may, for example, enable selective and/or graduated radial expansion at discrete locations along the distal region, providing enhanced control over the fitted transition profile and accommodating non-uniform anatomical or device geometries encountered during advancement.

Some embodiments may, for example, include a dilator with tension-responsive radial expansion module.

In some embodiments, a dilator may include a dilator body extending along a longitudinal axis from a proximal end to a distal end, with a tapered tip disposed at the distal end. An inner wall of the dilator body may define an inner lumen extending through the dilator body along the longitudinal axis. A tension-responsive radial expansion module may be disposed about the inner wall and may, for example, include at least one diagonally oriented member encapsulated by an outer wall. The tension-responsive radial expansion module may, for example, be selectively operable between an expanded state and a collapsed state by a selectively applied force aligned with the longitudinal axis, thereby enabling a physician or operator to control the radial profile of the dilator during a procedure.

In some embodiments, the at least one diagonally oriented member of the tension-responsive radial expansion module may, for example, include a braid configured to maintain a predetermined volumetric relationship. By virtue of this volumetric relationship, longitudinal compression of the braid may, for example, induce radial expansion of the tension-responsive radial expansion module, while longitudinal extension of the braid may, for example, induce radial contraction of the module. This braid-based configuration may, for example, provide a predictable and repeatable mechanical response to axially applied forces, enabling reliable control over the radial engagement profile of the dilator.

In some embodiments, the tension-responsive radial expansion module may, for example, be operated into the expanded state by application of an axial compressive force along the longitudinal axis, and may, for example, be operated into the collapsed state by application of an axial tensile force along the longitudinal axis. This push-to-expand, pull-to-collapse behavior may, for example, allow an operator to selectively engage or disengage the module with surrounding structures using intuitive, unidirectional force inputs delivered from the proximal end of the dilator.

In some embodiments, a longitudinal actuation member configured as a sleeve may be disposed about the tension-responsive radial expansion module. The sleeve-form actuation member may, for example, be configured to longitudinally compress the at least one diagonally oriented member, thereby inducing radial expansion of the tension-responsive radial expansion module. This sleeve arrangement may, for example, provide a compact, low-profile actuation mechanism that distributes compressive force circumferentially and uniformly about the module, promoting symmetric radial expansion.

In some embodiments, the tension-responsive radial expansion module may be disposed at a distal region of the dilator body, proximal of the tapered tip. Positioning the module at the distal region may, for example, place the radial expansion capability at or near the working end of the dilator, enabling targeted engagement with anatomical structures and/or catheter interfaces at a location where precise fitment may be helpful.

In some embodiments, the dilator body may be receivable within a lumen of a guide extension catheter, the lumen extending from a proximal end to a distal mouth. In this configuration, the tension-responsive radial expansion module may, for example, be operated into the expanded state to form a fitted transition between an outer surface of the dilator body and the distal mouth of the lumen of the guide extension catheter. This fitted transition may, for example, reduce or eliminate fish-mouthing at the distal mouth of the guide extension catheter, thereby facilitating smoother passage of the assembly through the vasculature and reducing the likelihood of snagging on anatomical structures and/or lesions.

In some embodiments, the tension-responsive radial expansion module may be further configured to be operated into the collapsed state to permit unobstructed passage of the dilator body through the lumen of the guide extension catheter during advancement and/or withdrawal. Once the tension-responsive radial expansion module is positioned at or proximal to the distal mouth of the guide extension catheter, the module may then, for example, be operated into the expanded state to establish the fitted transition at that location. This selective collapse-and-expand sequence may, for example, allow the dilator to be advanced through the guide extension catheter with a low profile and then selectively engaged at the distal mouth to achieve the desired fitment without requiring removal or exchange of the device.

In some embodiments, the at least one diagonally oriented member may include a braided mesh sleeve configured to contract longitudinally while expanding radially. This braided mesh geometry may, for example, provide selectively controlled engagement with an inner surface of a guide extension catheter, enabling an operator to modulate the degree of radial contact force applied to the catheter wall. Such embodiments may, for example, allow for fine-tuned fitment across guide extension catheters having varying lumen diameters, while the mesh structure maintains structural integrity and flexibility during navigation through tortuous anatomy.

Some embodiments may, for example, include a thin-walled guide liner.

In some embodiments, a guide liner may be provided in the form of a tubular body extending along a longitudinal axis from a proximal end to a distal end, the tubular body having a wall defining a lumen therethrough. The tubular body may include a proximal body region having a relatively larger lumen cross-sectional area and a distal region having a relatively smaller lumen cross-sectional area, with both cross-sectional areas being taken perpendicular to the longitudinal axis. The distal region may, for example, be configured to be plastically radially expanded during an initial passage of a dilator through the distal region, such that the smaller lumen cross-sectional area of the distal region increases toward the larger lumen cross-sectional area of the proximal body region. This configuration may, for example, allow the guide liner to be introduced through anatomy in a low-profile state and then expanded in situ to a working diameter upon first passage of the dilator, reducing the force required for initial advancement while ultimately providing a full-diameter working channel.

In some embodiments, the wall of the tubular body at the distal region may have a wall thickness of less than 0.005 inches. Such a thin-walled construction at the distal region may, for example, reduce the overall profile of the guide liner during advancement through vasculature, while still permitting the distal region to be plastically expanded by the dilator during initial passage.

In some embodiments, the wall thickness at the distal region may be less than or equal to 0.004 inches. This reduced wall thickness may, for example, further minimize the crossing profile of the guide liner, which may be particularly advantageous when navigating through tight and/or heavily calcified lesions.

In some embodiments, the wall thickness at the distal region may be less than or equal to 0.0025 inches. Such an extremely thin-walled distal region may, for example, provide maximum lumen diameter for a given outer profile, which may facilitate passage of devices through the guide liner following expansion.

In some embodiments, the distal region of the tubular body may be formed from a material selected from polytetrafluoroethylene (PTFE), polyetheretherketone (PEEK), and/or high molecular weight high density polyethylene (HMWHDPE). These materials may, for example, provide a combination of low friction, biocompatibility, and sufficient plasticity to permit controlled radial expansion of the distal region upon initial dilator passage.

In some embodiments, the distal region may include a first polymer having a first friction coefficient and a second polymer having a lower elastic modulus than the first polymer. The second polymer may, for example, contribute a radially stretchable characteristic to the distal region. This multi-polymer construction may, for example, allow the distal region to undergo controlled plastic deformation during dilator passage while the first polymer maintains a low-friction luminal surface to facilitate device advancement.

In some embodiments, the distal region may be configured to be plastically radially expanded not only by passage of the dilator therethrough, but additionally (or alternatively) by inflation of a balloon disposed within the lumen at the distal region. This dual-expansion approach may, for example, provide a supplemental or staged expansion mechanism, which may be useful when the dilator alone is insufficient to fully expand the distal region to a desired working diameter, such as in the presence of a particularly resistant and/or calcified lesion.

In some embodiments, the proximal body region may have a lumen diameter corresponding to approximately 6 French and the distal region may have a lumen diameter corresponding to approximately 5 French prior to the initial passage of the dilator. Upon initial passage of the dilator, the distal region may, for example, be configured to expand to a lumen diameter corresponding to approximately 6 French or greater. This size relationship may, for example, allow the guide liner to be advanced through anatomy in a reduced 5 French profile at its distal end while ultimately providing a 6 French or larger working channel following expansion, facilitating subsequent device delivery.

In some embodiments, the guide liner may be provided together with a dilator receivable within the lumen of the tubular body. The dilator may include a dilator body extending along the longitudinal axis and a tapered tip disposed at a distal end of the dilator body. The dilator body may have an outer diameter configured to plastically radially expand the distal region of the tubular body during the initial passage of the dilator through the distal region. This matched dilator-and-liner configuration may, for example, help ensure that the dilator is appropriately sized to reliably expand the distal region to a desired working diameter in a single pass.

In some embodiments, the dilator body may be configured to radially support the wall of the tubular body at the distal region to reduce or prevent axial collapse of the distal region during advancement through vasculature. Because the distal region has a thin wall and a reduced lumen cross-sectional area, it may be susceptible to axial buckling and/or collapse during advancement through tortuous and/or resistive anatomy. The dilator body, when disposed within the lumen, may, for example, provide an internal scaffold that maintains the shape and patency of the distal region during navigation.

In some embodiments, the distal region of the tubular body may have a length greater than 10 mm. A distal region of this length may, for example, provide a sufficient zone of reduced-profile construction to facilitate advancement through an extended segment of resistive anatomy before expansion.

In some embodiments, the distal region may have a length between 10 cm and 15 cm. This extended length may, for example, be advantageous in peripheral vascular procedures where a longer low-profile distal segment is used to traverse extended lesions and/or tortuous vessel segments prior to expansion.

In some embodiments, the tubular body may be configured as a guide extension catheter. In this configuration, the guide liner may, for example, function as a guide extension catheter having a thin-walled, plastically expandable distal region, which may combine the benefits of a low-profile delivery configuration with the working-channel functionality of a conventional guide extension catheter following in situ expansion.

Some embodiments may, for example, include intravascular dilator systems.

In some embodiments, an intravascular dilator system may include a guide extension catheter having a lumen extending from a proximal end to a distal mouth, where the guide extension catheter may include a distal region having a wall thickness of less than 0.005 inches and a lumen cross-sectional area at the distal region that is smaller than the lumen cross-sectional area at a proximal body region of the guide extension catheter. The system may further include a dilator receivable within the lumen of the guide extension catheter. The dilator may include a dilator body extending along a longitudinal axis from a proximal end to a distal end, a tapered tip disposed at the distal end of the dilator body, and a longitudinally-extending relief extending along a length of the dilator body such that a transverse cross-section of the dilator body taken perpendicular to the longitudinal axis is less than a full circle. The longitudinally-extending relief may, for example, be configured to accommodate an axial translation member disposed along an outer surface of the dilator body. The dilator body may, for example, be configured to plastically radially expand the distal region of the guide extension catheter during an initial passage of the dilator through the distal region, such that the lumen cross-sectional area at the distal region increases toward the lumen cross-sectional area at the proximal body region. This combined configuration may, for example, allow the system to be advanced through anatomy in a low-profile state, with the dilator simultaneously expanding the thin-walled distal region of the guide extension catheter to a working diameter while the longitudinally-extending relief accommodates the axial translation member without increasing the overall system profile.

In some embodiments, the dilator body may, for example, be configured to form a fitted transition between the outer surface of the dilator body and the distal mouth of the lumen of the guide extension catheter. This fitted transition may, for example, reduce or eliminate fish-mouthing at the distal mouth of the guide extension catheter, which may reduce the likelihood of the catheter snagging on vessel walls and/or lesions during advancement.

In some embodiments, the system may additionally include the axial translation member disposed within the longitudinally-extending relief of the dilator body. The axial translation member may, for example, include a hypotube configured to engage the proximal end of the dilator body to transmit a pushing force to advance the dilator body through the guide extension catheter. Disposing the axial translation member within the longitudinally-extending relief may, for example, help maintain a low overall system profile while providing a robust push-force transmission pathway to the dilator body.

In some embodiments, an intravascular dilator system may include a guide extension catheter having a lumen extending from a proximal end to a distal mouth, and a dilator receivable within the lumen of the guide extension catheter. The dilator may include a dilator body extending along a longitudinal axis from a proximal end to a distal end, the dilator body having an outer wall and an inner wall defining a guidewire lumen extending through the dilator body. A tapered tip may be disposed at the distal end of the dilator body. A unitary bladder may define a closed void disposed between the outer wall and the inner wall of the dilator body, the closed void being fluidly isolated from the guidewire lumen. The unitary bladder may extend radially from the dilator body at a distal region of the dilator body proximal of the tapered tip, and may be configured to self-adjust the outer diameter of the dilator body to form a fitted transition between the outer wall of the dilator body and the distal mouth of the lumen of the guide extension catheter. This self-adjusting bladder configuration may, for example, allow the dilator to passively conform to guide extension catheters having different lumen diameters, reducing or eliminating fish-mouthing without requiring active operator intervention, while the fluidly isolated guidewire lumen preserves an unobstructed pathway for guidewire passage.

In some embodiments, the guide extension catheter of the system described above may include a distal region having a wall thickness of less than 0.005 inches and a lumen cross-sectional area at the distal region that is smaller than the lumen cross-sectional area at a proximal body region of the guide extension catheter. The dilator body may be configured to radially expand the distal region of the guide extension catheter during passage of the dilator through the distal region. This combination may, for example, integrate the self-adjusting fitted-transition functionality of the unitary bladder with the in situ lumen-expansion capability of the thin-walled distal region, providing both a smooth transition at the distal mouth and progressive dilation of the guide extension catheter during initial advancement.

In some embodiments, the system may additionally include support elements disposed within the closed void of the unitary bladder. The support elements may, for example, be configured to reduce or prevent collapse of the guidewire lumen while permitting controlled deformation of the unitary bladder to adjust the outer diameter of the dilator body. The support elements may, for example, help maintain the patency of the guidewire lumen under compressive forces encountered during advancement through tight lesions and/or tortuous anatomy, while still allowing the bladder to deform radially as needed to achieve a fitted transition with the guide extension catheter.

In some embodiments, an intravascular dilator system may include a guide extension catheter having a lumen extending from a proximal end to a distal mouth, and a dilator receivable within the lumen of the guide extension catheter. The dilator may include a dilator body extending along a longitudinal axis from a proximal end to a distal end, a tapered tip disposed at the distal end of the dilator body, and an inner wall of the dilator body defining an inner lumen extending through the dilator body. A tension-responsive radial expansion module may be disposed about the inner wall and may, for example, include at least one diagonally oriented member disposed about the inner wall and an outer wall encapsulating the at least one diagonally oriented member. The system may further include a longitudinal actuation member configured to apply a force aligned with the longitudinal axis to the tension-responsive radial expansion module to selectively operate the module between an expanded state and a collapsed state. In the expanded state, the tension-responsive radial expansion module may, for example, form a fitted transition between an outer surface of the dilator body and the distal mouth of the lumen of the guide extension catheter. In the collapsed state, the dilator body may be translatable through the lumen of the guide extension catheter. This selectively actuated configuration may, for example, allow an operator to actively control when the dilator engages the guide extension catheter in a fitted manner, enabling low-profile advancement in the collapsed state and secure, fish-mouth-reducing engagement in the expanded state as desired during the procedure.

In some embodiments, the at least one diagonally oriented member of the tension-responsive radial expansion module may include a braid. Application of an axial compressive force by the longitudinal actuation member may, for example, operate the tension-responsive radial expansion module into the expanded state, while application of an axial tensile force by the longitudinal actuation member may, for example, operate the module into the collapsed state. The braid geometry may, for example, translate axial compressive loading into radial expansion in a predictable and controlled manner, consistent with the mechanical behavior of braided structures, while axial tension may cause the braid to elongate and radially contract, returning the module to its low-profile collapsed state for repositioning and/or withdrawal.

It will be understood that various modifications can be made within the scope of this disclosure. For example, one or more advantageously results may be achieved if components are removed, added, multiplied, scaled, and/or rearranged, and/or if steps in a method are omitted, added, repeated, and/or performed in a different order. Therefore, other implementations are contemplated within the scope of the following claims.

What is claimed is:

1. A lumen-fitted dilator, comprising:
a dilator body extending along a longitudinal axis from a proximal end to a distal end, the dilator body having an outer surface;
a tapered tip disposed at the distal end of the dilator body; and
a lumen fitment module disposed on the outer surface of the dilator body, the lumen fitment module having an adjustable outer diameter configured to transition between a first outer diameter and a second outer diameter different from the first outer diameter, the lumen fitment module configured to form a fitted transition between the outer surface of the dilator body and a distal mouth of a lumen of a guide extension catheter when the dilator body is received within the guide extension catheter,
wherein the adjustable outer diameter of the lumen fitment module is configured to interface with a plurality of guide extension catheters having different lumen diameters.

2. The lumen-fitted dilator of claim 1, wherein the lumen fitment module is configured to fill the lumen of the guide extension catheter at the distal mouth such that the dilator body is urged into circumferential contact with the guide extension catheter.

3. The lumen-fitted dilator of claim 2, wherein the lumen fitment module comprises a hub disposed proximally to the tapered tip, the hub having a cross-sectional geometry sized to substantially occlude the lumen of the guide extension catheter.

4. The lumen-fitted dilator of claim 3, wherein the hub comprises a cross-sectional shape defining a longitudinal relief configured to accommodate an axial translation member junction point.

5. The lumen-fitted dilator of claim 2, wherein the lumen fitment module is compressible such that the lumen fitment module compresses when received within the lumen of the guide extension catheter and expands when advanced beyond the distal mouth of the guide extension catheter.

6. The lumen-fitted dilator of claim 2, wherein the lumen fitment module comprises a self-expanding region disposed proximally to the tapered tip, the self-expanding region configured to radially expand toward an inner wall of the guide extension catheter when the dilator body is disposed within the guide extension catheter.

7. The lumen-fitted dilator of claim 1, further comprising an axial translation member extending proximally from the proximal end of the dilator body, the axial translation member comprising a hypotube configured to engage the proximal end of the dilator body to transmit a pushing force to advance the dilator body through the guide extension catheter.

8. The lumen-fitted dilator of claim 7, wherein the dilator body has a wall thickness of 0.004 inches or less.

9. The lumen-fitted dilator of claim 7, wherein the axial translation member further comprises a push rod coupled to the hypotube, the hypotube disposed at a distal end of the push rod.

10. The lumen-fitted dilator of claim 1, wherein the lumen fitment module comprises a bladder disposed between an outer wall and an inner wall of the dilator body, the bladder configured to deform to adjust the outer diameter of the lumen fitment module.

11. The lumen-fitted dilator of claim 10, wherein the bladder comprises support elements disposed within the bladder.

12. The lumen-fitted dilator of claim 1, wherein the lumen fitment module comprises a braid configured to maintain a predetermined volumetric relationship, the braid configured to expand radially upon longitudinal compression of the braid by a longitudinal actuation member.

13. The lumen-fitted dilator of claim 1, wherein the dilator body comprises polytetrafluoroethylene (PTFE), polyetheretherketone (PEEK), fluorinated ethylene propylene (FEP), ethylene tetrafluoroethylene (ETFE), polyethylene, and/or polyamide.

14. The lumen-fitted dilator of claim 1, wherein the plurality of guide extension catheters having different lumen diameters comprises guide extension catheters ranging from 5 French to 7 French.

15. The lumen-fitted dilator of claim 1, wherein the dilator body comprises a multi-durometer polymer construction comprising a proximal portion having a first durometer and a distal portion having a second durometer less than the first durometer.

16. An intravascular dilator system, comprising:
a guide extension catheter having a lumen extending from a proximal end to a distal mouth; and
a dilator receivable within the lumen of the guide extension catheter, the dilator comprising a dilator body extending along a longitudinal axis from a proximal end to a distal end, a tapered tip disposed at the distal end of the dilator body, and a lumen fitment module disposed on an outer surface of the dilator body, the lumen fitment module having a cross-sectional shape defining a longitudinal relief configured to accommodate an axial translation member disposed along the outer surface of the dilator body.

17. The intravascular dilator system of claim 16, wherein the lumen fitment module is configured to form a fitted transition between the outer surface of the dilator body and the distal mouth of the lumen of the guide extension catheter such that the dilator body is urged into circumferential contact with the guide extension catheter.

18. The intravascular dilator system of claim 17, wherein the lumen fitment module comprises a hub disposed proximally to the tapered tip, the hub having a cross-sectional geometry sized to occlude the lumen of the guide extension catheter at the distal mouth.

19. The intravascular dilator system of claim 18, further comprising the axial translation member extending proximally from the proximal end of the dilator body, the axial translation member comprising a hypotube configured to engage the proximal end of the dilator body to transmit a pushing force to advance the dilator body through the guide extension catheter.

20. The intravascular dilator system of claim 19, wherein the lumen fitment module is configured to interface with a plurality of guide extension catheters having different lumen diameters such that the dilator is operable with each of the plurality of guide extension catheters.
